SEQUENCE LISTING (11 sequences)

SEQ ID NO: 1 - protein, 1040 aa, Homo sapiens
MGEEGGSASHDEEERASVLLGHSPGCEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL

SEQ ID NO: 2 - protein, 953 aa, Homo sapiens
MEEQGHSEMEIIPSESHPHIQLLKSNRELLVTHIRNTQCLVDNLLKNDYFSAEDAEIVCACPTQPDKVRKILDLVQSKGEEVSEFFLYLLQQLADAYVDLRPWLLEIGFSPSLLTQSKVVVNTDPVSRYTQQLRHHLGRDSKFVLCYAQKEELLLEEIYMDTIMELVGFSNESLGSLNSLACLLDHTTGILNEQGETIFILGDAGVGKSMLLQRLQSLWATGRLDAGVKFFFHFRCRMFSCFKESDRLCLQDLLFKHYCYPERDPEEVFAFLLRFPHVALFTFDGLDELHSDLDLSRVPDSSCPWEPAHPLVLLANLLSGKLLKGASKLLTARTGIEVPRQFLRKKVLLRGFSPSHLRAYARRMFPERALQDRLLSQLEANPNLCSLCSVPLFCWIIFRCFQHFRAAFEGSPQLPDCTMTLTDVFLLVTEVHLNRMQPSSLVQRNTRSPVETLHAGRDTLCSLGQVAHRGMEKSLFVFTQEEVQASGLQERDMQLGFLRALPELGPGGDQQSYEFFHLTLQAFFTAFFLVLDDRVGTQELLRFFQEWMPPAGAATTSCYPPFLPFQCLQGSGPAREDLFKNKDHFQFTNLFLCGLLSKAKQKLLRHLVPAAALRRKRKALWAHLFSSLRGYLKSLPRVQVESFNQVQAMPTFIWMLRCIYETQSQKVGQLAARGICANYLKLTYCNACSADCSALSFVLHHFPKRLALDLDNNNLNDYGVRELQPCFSRLTVLRLSVNQITDGGVKVLSEELTKYKIVTYLGLYNNQITDVGARYVTKILDECKGLTHLKLGKNKITSEGGKYLALAVKNSKSISEVGMWGNQVGDEGAKAFAEALRNHPSLTTLSLASNGISTEGGKSLARALQQNTSLEILWLTQNELNDEVAESLAEMLKVNQTLKHLWLIQNQITAKGTAQLADALQSNTGITEICLNGNLIKPEEAKVYEDEKRIICF

SEQ ID NO: 3 - protein, 317 aa, Homo sapiens
PAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL

SEQ ID NO: 4 - protein, 373 aa, Homo sapiens
NKDHFQFTNLFLCGLLSKAKQKLLRHLVPAAALRRKRKALWAHLFSSLRGYLKSLPRVQVESFNQVQAMPTFIWMLRCIYETQSQKVGQLAARGICANYLKLTYCNACSADCSALSFVLHHFPKRLALDLDNNNLNDYGVRELQPCFSRLTVLRLSVNQITDGGVKVLSEELTKYKIVTYLGLYNNQITDVGARYVTKILDECKGLTHLKLGKNKITSEGGKYLALAVKNSKSISEVGMWGNQVGDEGAKAFAEALRNHPSLTTLSLASNGISTEGGKSLARALQQNTSLEILWLTQNELNDEVAESLAEMLKVNQTLKHLWLIQNQITAKGTAQLADALQSNTGITEICLNGNLIKPEEAKVYEDEKRIICF

SEQ ID NO: 5 - protein, 441 aa, Homo sapiens [sequence omitted]

SEQ ID NO: 6 - protein, 570 aa, Homo sapiens
KEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRT

SEQ ID NO: 7 - protein, 334 aa, Homo sapiens [sequence omitted]

SEQ ID NO: 8 - DNA, 31 bp, Artificial
  synthetic polymerase chain reaction primer sequence
caccatgaacaaggatcacttccagttcacc

SEQ ID NO: 9 - DNA, 27 bp, Artificial
  synthetic polymerase chain reaction primer sequence
tcagaaacagataatccgcttctcatc

SEQ ID NO: 10 - DNA, 32 bp, Artificial
  synthetic polymerase chain reaction primer sequence
caccatgaccatgccagctgcaccgggtgagg

SEQ ID NO: 11 - DNA, 29 bp, Artificial
  synthetic polymerase chain reaction primer sequence
tcaaagcaagagtctggtgtccctgcagc

(19) United States
(12) Patent Application Publication
(10) Pub. No.: US 2010/0286027 A1
(43) Pub. Date: Nov. 11, 2010

(54) ANTI-BACTERIAL COMPOSITIONS

(75) Inventors: Scott Parkinson, Horsham (GB); Laurent-Herve Perez, Colombier (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/812,120

(22) PCT Filed: Jan. 13, 2009
(86) PCT No.: PCT/EP09/50317

§ 371 Date: Jul. 8, 2010

(30) Foreign Application Priority Data
Jan. 15, 2008 (EP) 08150290.8

(51) Int. Cl. A61K 38/02; C07K 14/00; C07K 14/435; A61P 31/04; A61P 1/00

(52) U.S. Cl. 514/2.4; 530/350

(57) ABSTRACT

The invention relates to an isolated protein for use as an antimicrobial agent comprises a plurality of LRR (leucine rich repeat) domains, each LRR domain independently comprising an amino acid sequence of formula (I): (F1LxxLxL(xxZ)_{Y}F2) wherein: F1 and F2 are independently, a contiguous amino acid sequence of between 1 and 30 residues; x can be any amino acid; L can be Leu, Ile, Val or Phe; Z can be NxL or CxxL; N is Asn, Thr, Ser or Cys; C is Cys or Ser; and Y=0 or 1.

### 1. FIELD OF THE INVENTION

The present invention relates to anti-bacterial compositions and methods of treating or preventing pathogenic bacterial infections. More particularly, the present invention relates to anti-bacterial pharmaceutical compositions, for the treatment or prevention of bacterial infections and diseases associated therewith. Other aspects, objects and advantages of the present invention will be apparent from the description below.

### 2. BACKGROUND OF THE INVENTION

The intestinal epithelium ring-fences bacteria in the gut lumen allowing the host to harvest prokaryotic metabolites it cannot synthesize itself while protecting it from infection. Due to their constant exposure to the microbiota of the gastrointestinal tract, epithelial cells are also the primary point of entry for many pathogens. In order to prevent infection of the host, epithelial cells express various pattern-recognition receptors (PRR), like Nod2, to provide a first line of defence against invasion. PRRs are essential components of the innate immune system. They recognise conserved motifs found in bacteria, oomycetes, nematodes, fungi, viruses and insects and trigger an immediate measured and targeted response in the host to the invading microorganism. (Ting J P Y and Davis B K, 2005).

A common element of many PRRs, including the Nod, Nalp and plant R protein families, is a leucine-rich repeat (LRR) domain. While the conserved leucine-rich repeat provides the structural scaffold for the iconic horseshoe shape of the LRR domain the PRR flanking regions are diverse polypeptide segments that confer recognition of common microbial motifs (Matsushima N. et al., 2005). The LRR domains are found in PRRs from plants to humans and are essential for resistance of the host to pathogens. Deletion or spontaneous mutation of specific LRR-containing proteins confers susceptibility of the host to infection (Dangl J L and Jones J D G, 2001). Agnathan fish have exploited the LRR domain as a scaffold to develop a novel adaptive immune system based on recombination of individual LRR peptide sequences (Pancer Z et al., 2004, Alder M N et al., 2005, Nagawa F et al., 2007).

In humans, genetic studies have identified single nucleotide polymorphisms (SNPs) in many LRRs that are associated with susceptibility to various diseases including those of infectious or inflammatory origin (Matsushima N., et al, 2005). Nod2 is perhaps the most extensively studied of the disease-associated LRR-containing proteins. It confers susceptibility to Crohn's disease and its association with the disease has been confirmed in numerous independent studies (Hugot J P et al., 2001, Ogura Y et al., 2001, Hampe J et al., 2007, Libioulle C et al., 2007, Raelson J V et al., 2007, The Wellcome Trust Case Control Consortium, 2007). Nod2 mutations in the LRR domain confer susceptibility to Crohn's while specific mutations in the adjacent NACHT domain of Nod2 are the genetic cause of Blau syndrome; a rare autosomal dominant disorder characterized by early-onset granulomatous arthritis, uveitis, and skin rash with camptodactyly (Miceli-Richard C et al., 2001). This suggests that a specific molecular function for the Nod2 LRR domain confers susceptibility to intestinal disease.

Most research surrounding Nod2 has focused on its activation of signal transduction pathways in response to putative ligands. The three Nod2 SNPs most commonly associated with Crohn's disease are all deficient in their response to MDP (a component of the bacterial proteoglycan coat) and demonstrate a lack of NFkB translocation and production of cytokines (Barnich N et al., 2005). In contrast, Crohn's disease is characterised by elevated NFkB-dependent cytokine production. Debate about whether Crohn's-associated Nod2 SNPs are gain or loss of function mutations is ongoing (Watanabe T et al., 2004, Kobayashi K S et al., 2005, Maeda S, 2005).

Nod2's role in protecting the host against infection by bacteria has also been highlighted in studies using a Nod2 knockout mouse strain (Kobayashi K S et al., 2005). Nod2 knockouts were more susceptible to oral (but not systemic) infection by Listeria monocytogenes. This is an important observation, since Crohn's patients have been reported to demonstrate a substantial increase in their intracellular and epithelium-associated bacteria (Swidsinski A et al., 2002, Darfeuille-Michaud A, 2002, Liu Y et al., 1995). Some reports have suggested a role for Nod2 in preventing bacterial infection of cells (Hisamatsu T et al., 2003). These studies indicated a deficiency of the Crohn's-associated Nod2 3020insC protein to function as a defensive factor against intracellular bacteria. A follow-up study by the same group indicated a dependency on the mitochondrial protein grim19 for Nod2-dependent protection against Salmonella infection (Barnich N et al., 2005). Other members of the Nod family (Nod1) have also demonstrated a protective function against intracellular bacteria (Zilbauer M et al., 2007, Travassos L H et al., 2005). In contrast to Nod2, Nod1 does not associate with grim19 (Barnich N et al., 2005) suggesting the mechanism by which Nod proteins prevent infection by bacteria remains to be determined.

All references disclosed in the present specification, including any specification from which this application claims priority, are expressly and entirely incorporated herein by reference.

### 3. SUMMARY OF THE INVENTION

The present invention is based, at least in part, on a finding that proteins containing a leucine rich repeat (LRR) motif have a direct anti-bacterial activity.

In one aspect of the invention there is provided an isolated protein comprising (or consisting essentially of, or consisting of) an LRR of formula (I):

(F1LxxLxLxxZF2) (I)

### Wherein

F1 and F2 are independently, a contiguous amino acid sequence of between 1 and 30 residues;
x can be any amino acid,

### L can be Leu, Ile, Val or Phe;

### Z can be NxL or CxxL;

### N is Asn, Thr, Ser or Cys;

### C is Cys or Ser;

In another aspect of the invention, there is provided an isolated protein comprising (or consisting essentially of, or consisting of) in tandem two or more, (e.g. between two and fifty) LRRs of formula (I).

In another aspect of the invention there is provided an isolated protein comprising (or consisting essentially of or consisting of) two or more (e.g. between two and fifty) LRRs (e.g. in tandem) of formula (I) derived from a naturally occurring LRR containing protein.

In another aspect of the invention, there is provided an isolated protein comprising (or consisting essentially of, or consisting of) a nucleotide binding site (NBS)-LRR, such as a NOD-LRR (e.g. NOD2-LRR or NOD1-LRR, particularly human NOD2-LRR or human NOD1-LRR). In other aspects, there is provided an isolated protein comprising (or consisting essentially of, or consisting of) a CIITA-LRR, a Toll receptor-LRR (such as TLR2,4,5,7,8,9-LRR domain), a NAIP-LRR.

In one aspect of the invention there is provided an isolated protein comprising (or consisting essentially of, or consisting of) a nucleotide-binding oligomerization domain (NOD), an amino terminal effector domain and a carboxyl terminal leucine rich repeat (LRR) domain.

In another aspect of the invention there is provided a composition (particularly a pharmaceutical composition having anti-bacterial activity) comprising (for example as its sole active ingredient) an isolated protein comprising (or consisting essentially of, or consisting of) a NOD domain, an amino terminal death fold domain (such as CARD, Pyrin, death domain or death effector domain) and a carboxyl terminal LRR domain.

In another aspect of the invention there is provided an isolated protein comprising (or consisting essentially of, or consisting of) a NOD domain, an amino terminal caspase recruitment domain (CARD) and a carboxyl terminal LRR domain.

In another aspect of the invention there is provided a composition comprising (for example as its sole active ingredient) an isolated protein comprising or consisting essentially of a NOD domain, an amino terminal CARD domain and a carboxyl terminal LRR domain.

In yet another aspect of the invention there is provided a composition, particularly a pharmaceutical composition comprising (e.g. as its sole active ingredient) an isolated NOD protein, particularly NOD1 and/or NOD2 and more particularly human NOD1 and/or human NOD2.

In yet another aspect of the invention there is provided a composition, particularly a pharmaceutical composition (such as a bactericidal pharmaceutical composition) comprising (e.g. as its sole active ingredient) an isolated TLR protein, particularly a mammalian TLR protein and more particularly a human TLR protein such as human TLR2 and/or human TLR4 and/or human TLR5.

In another aspect there is provided an anti-bacterial (e.g. bactericidal) composition (particularly a pharmaceutical composition) comprising (for example as its sole active ingredient) an isolated NOD2 protein (particularly human NOD2).

In another aspect of the invention there is provided a pharmaceutical composition comprising an isolated protein comprising (or consisting essentially of, or consisting of) a NOD domain, an amino terminal death fold domain (such as a CARD domain) and a carboxyl terminal LRR domain together with a pharmaceutically acceptable carrier.

In another aspect of the invention there is provided a pharmaceutical composition (particularly a bactericidal pharmaceutical composition comprising (e.g. as its sole active ingredient) an isolated NOD protein (such as human NOD1 or human NOD2), particularly isolated human NOD2 and a pharmaceutically acceptable carrier.

In another aspect of the invention there is provided a method of/for treating or preventing a pathogen infection (particularly bacterial infection) which method comprises providing a composition comprising an isolated protein comprising (or consisting essentially of, or consisting of) a NOD domain, an amino terminal death fold domain (such as a CARD domain) and a carboxyl terminal LRR domain.

In another aspect of the invention there is provided a method of/for treating or preventing a pathogen infection (particularly bacterial infection) which method comprises providing a composition comprising an isolated protein comprising (or consisting essentially of, or consisting of) a NOD domain, an amino terminal death fold domain (such as a CARD domain) and a carboxyl terminal LRR domain.

In another aspect of the invention there is provided a method of/for treating or preventing a pathogen infection (such as a bacterial infection) which method comprises providing a composition comprising an isolated NOD protein, such as isolated human NOD1 and/or human NOD2.

In another aspect of the invention there is provided a method of/for treating or preventing a pathogen infection, particularly bacterial infection in a human patient which method comprises administering to said patient (a pharmaceutical composition comprising) a therapeutically effective amount of an isolated NOD protein, particularly isolated human NOD1 and/or human NOD2.

In another aspect of the invention there is provided the use of an isolated protein which protein comprises a NOD domain, an amino terminal death fold domain (such as CARD) and a carboxyl terminal LRR domain in medicine, particularly human medicine.

In another aspect of the invention there is provided the use of an isolated protein (such as isolated human NOD2) which protein comprises a NOD domain, an amino terminal death fold domain (such as CARD) and a carboxyl terminal LRR in the manufacture of a medicament for the treatment or prevention of pathogen infection, particularly bacterial infection, more particularly gram positive bacterial infection.

In another aspect of the invention there is provided the use of an isolated protein which protein comprises a NOD domain, an amino terminal death fold domain (such as CARD) and a carboxyl terminal LRR domain in the manufacture of a medicament for the treatment of Crohns disease, Inflammatory bowel disease, septicaemia.

In another aspect of the invention there is provided the use of an isolated NOD protein (such as human NOD1 or human NOD2) in the manufacture of a medicament for the treatment of Crohns disease, Inflammatory bowel disease.

In another aspect of the invention there is provided a bactericidal pharmaceutical composition comprising a protein comprising (or consisting essentially of, or consisting of) a LRR domain (for example as its sole active ingredient) together with a pharmaceutically acceptable carrier. In one embodiment, the LRR domain is a human NOD-LRR such as human NOD1-LRR or human NOD2-LRR. In other embodiments, the LRR domain is a TLR-LRR domain such as a human TLR-LRR e.g. TLR2-LRR, TLR4-LRR, TLR5-LRR, TLR7-LRR, TLR8-LRR, TLR9-LRR.

Use of the protein and/or protein of the invention to kill bacteria, particularly gram positive bacteria is also contemplated.

In another embodiment, there is provided an isolated non-human mammalian LRR protein (such as a NOD or TLR protein) for use in treating and/or preventing pathogenic bacteria infection in the non-human mammal from which the LRR protein is derived.

In another aspect of the invention there is provided a method of/for identifying a bactericidal protein which method comprises contacting a bacteria, particularly a bacteria pathological to a mammal such as a human with an isolated LRR protein and identifying said protein if it demonstrates a bactericidal activity. In some embodiments, the bacteria is aerobic, in other embodiments anaerobic, in further other embodiments the bacteria gram positive or gram negative.

### 4. BRIEF DESCRIPTION OF THE DRAWINGS

FIG. 1: Immunohistochemical determination of Nod2 expression in colonic epithelium. Panel A: Formalin-fixed paraffin embedded segments of rat and human colon were probed with an affinity-purified rabbit anti-Nod2 antibody (AB5; left) or rabbit IgG (right) as a negative control. DAPI staining is indicated in purple. Panel B: Rat colon was extracted directly into SDS-PAGE sample buffer and analysed by Western blot using AB5. A single protein of approximately 100 kDa was identified correlating with Nod2.

FIG. 2: Immunolocalization of Nod2 following incubation of SW480 intestinal epithelial cells with E. coli. SW480 cells were incubated with or without E. coli at an MOI of 10000:1 for 4 hours. The cells were fixed and stained with anti-Nod2 (green), phalloidin (red) and DAPI (purple). Nod2 shifted from the cytosol to punctate structures in the cell cytoplasm following incubation with E. coli.

FIG. 3: Immunolocalization of Nod2 with E. coli in intestinal epithelial cells. Confluent monolayers of Caco2 intestinal epithelial cells were incubated with E. coli at an MOI of 10000:1 for 2 hours. Cells were fixed and analysed by immunofluorescence with anti-Nod2 (AB5) and anti-LPS antibodies using confocal microscopy.

FIG. 4: Aggregation of E. coli in vitro following incubation with recombinant Nod2 LRR domains. E. coli (10⁶) in 1 ml of PBS were incubated with either 20 microgram/ml BSA or purified recombinant Nod2 LRR domains for 12 hours. Aliquots of the cultures were inoculated on a coverslipped slide and analyzed by light microscopy using a 63× objective.

FIG. 5: Streptococcus pneumoniae infection of Nod2-expressing 293 cells. 293 cells stably expressing chloramphenicol acetyl transferase (control), Nod2 or a Crohn's-associated Nod2 mutant (Nod2-3020insC) from the same chromosomal locus were infected with Streptococcus pneumoniae (ATCC 49619) at an MOI of 10:1. The gentamycin protected bacteria were plated on chocolate agar to observe the number of intracellular bacteria in each cell line.

FIG. 6: Purified Nod2 LRR domains (Nod2: 30 microgram/ml) were preincubated with 200 microgram/ml of the indicated bacterial component prior to addition to Staphylococcus aureus. BSA was added as a protein control. Commercial proteoglycan extracts (sPGN: soluble proteoglycan, iPGN: insoluble proteoglycan), lipoteichoic acid (LTA: crude lipoteichoic acid extract, upLTA: ultrapure lipoteichoic acid extract), or heat-killed S. aureus (HKSA) were used. Control indicates bacterial growth in the presence of BSA only.

FIG. 7: Purification of Nod2 LRR antibacterial target (E. coli). E. coli (ATCC) was grown overnight in LB broth, pelleted and the bacterial pellet extracted by French press. A competition assay was performed monitoring Nod2 LRR domain activity versus Staphylococcus aureus (ATCC 29233). At each step, the volume of the fractions was made up to equal volume and samples added to the antibacterial assay. The inhibiting fraction was finally found in the detergent (NP40)-insoluble fraction. This fraction was extracted with guanidinium HCl, separated by gel filtration and individual fractions collected and assessed for inhibition of Nod2 LRR activity versus S. aureus. Fraction 5 (F5) contained protein(s) that inhibited LRR activity as determined by sensitivity to proteinase K.

FIG. 8: LRR affinity purification of Nod2 antibacterial target and identification by mass spectrometry. Panel A: Fraction 5 from the gel filtration of the guanidinium HCl-extracted detergent-insoluble E. coli fraction in FIG. 2 was loaded onto a Nod2 LRR domain affinity column. Bound proteins were eluted by an NaCl gradient. Panel B: Coomassie-stained gel of Fraction 5 (F5) prior to Nod2 LRR domain affinity purification and the salt-eluted fractions (E) from the affinity column. Panel C: Mass spectrometer protein identifications in extracted bands as indicated in panel B.

FIG. 9: Separation of wild type and 3020insC LRR domain affinity-purified detergent-insoluble proteins from E. coli. E. coli were fractionated by French press, centrifuged and the pellet extracted with guanidinium HCl. The solubilised pellet was split into two and each fraction separated on either a Nod2 LRR (WT) or Nod2 3020insC LRR (3020) affinity column. Proteins associated on either column were eluted with salt, precipitated with either cold acetone or TCA/acetone and separated by SDS PAGE gel electrophoresis. Individual regions of the gel were selected, excised and processed for mass spectrometer identification of proteins (as indicated in Table 4, Table 5).

FIG. 10: TLR2 and Nalp3 LRR domains inhibit L. monocytogenes viability as demonstrated by ATP-coupled luminescence assay. L. monocytogenes (5×10⁵ bacteria/100 μl) were incubated with increasing concentrations of the indicated recombinant LRR domains for 6 hours at 37° C. and ATP levels assessed by luminescence assay (BacTiter-Glo: Promega). Values shown are relative to controls incubated in the absence of LRR domains (100%). Results are representative of two experiments for TLR2 and Nalp3.

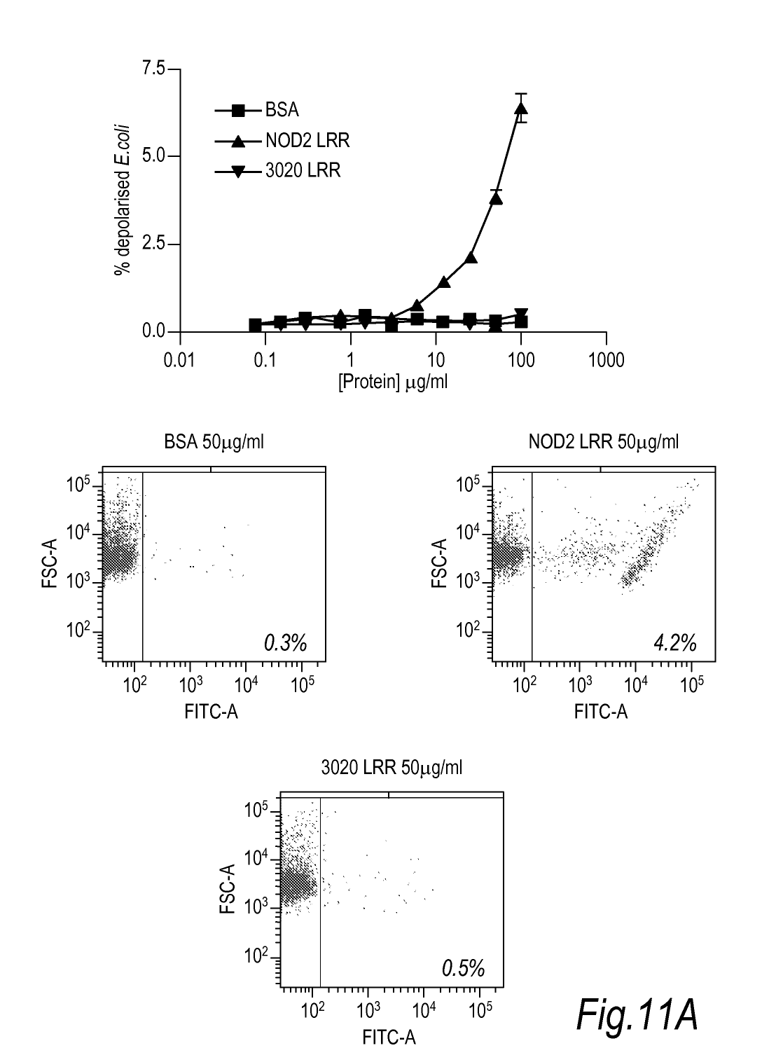

FIG. 11: Bacterial killing by purified Nod2 LRR domains is deficient in protein carrying the Crohn's-associated Nod2 3020insC mutation. Results shown are all representative of several experiments. Panels A and B: Nod2 LRR domains influence the membrane polarity of E. coli (Panel A) and B. subtilis (Panel B). Proteins were added at the concentration indicated to 5×10⁵ bacteria in 100 μL growth medium and incubated for 2 hours at 37° C. 15 minutes prior to the end of the time course, 50 μl of 10 μg/ml DiBAC4 solution was added to each well. Plates were washed twice with 750 μl ice cold PBS/well. The percentage of depolarised bacteria taking up the dye was determined by flow cytometry. Panel C: B. subtilis membrane polarity is influenced by the LRR domains from a range of pattern-recognition receptors. Bacteria were treated with the indicated LRR domains as described for Panels A and B and their effect on the membrane polarity of the bacteria was quantified. Panel D: Anti-bacterial activity of Nod1 and Nod2 but not Nod2 3020insC LRR domains demonstrated by agar diffusion assay. Agar plates were inoculated with a lawn of the indicated bacteria. Approximately 0.5 cm diameter holes were punched into the agar with a sterile glass pipette and the indicated protein (BSA protein control or indicated LRR domain) or antibiotic (ampicillin or kanamycin) added to each well at a concentration of 0.5 mg/ml in sterile PBS.

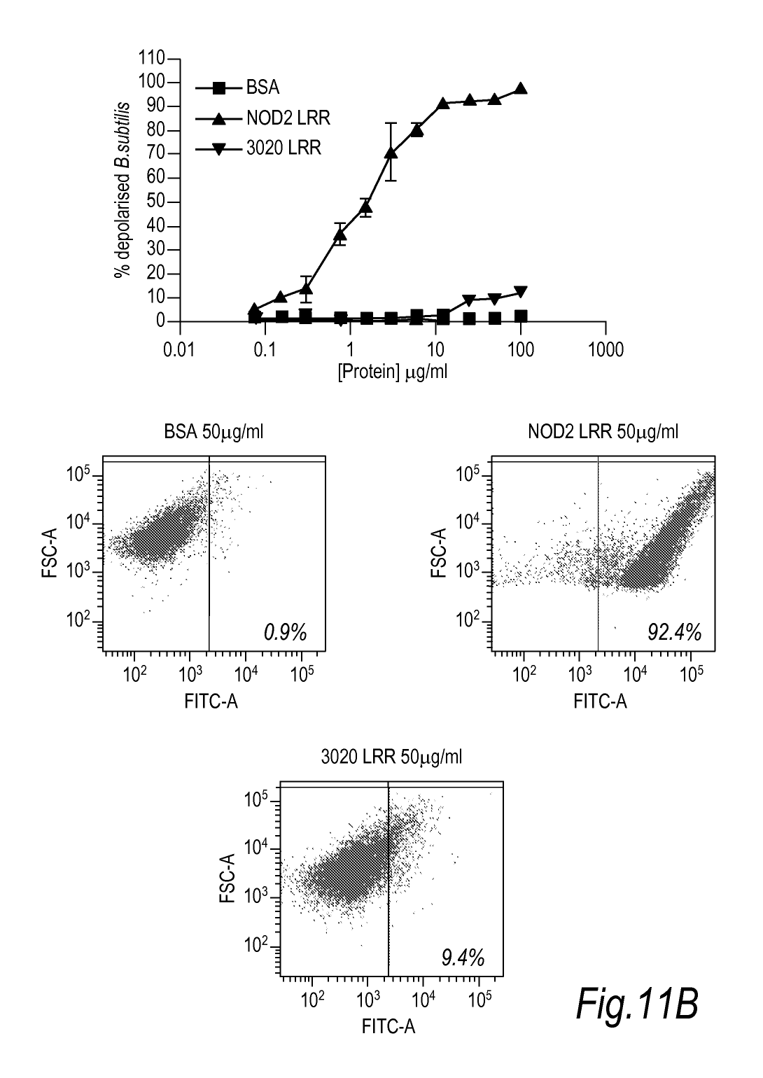

FIG. 12: Nod2 SNPs (full length) inhibit B. subtilis, S. aureus, L. monocytogenes and E. faecals viability as demonstrated by ATP-coupled luminescence assay. This activity is deficient in protein carrying the Crohn's-associated Nod2 3020insC and G908R mutations.

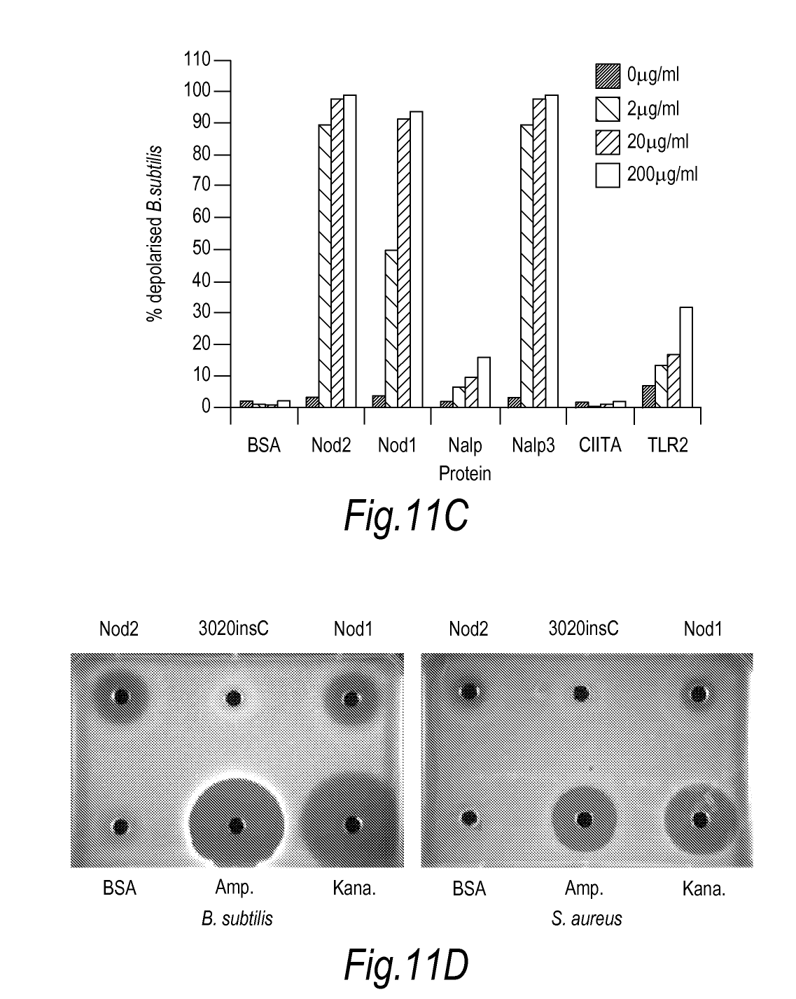

FIG. 13: The effect of Nod2 on S. aureus viability as demonstrated by ATP-coupled luminescent assay was tested under conditions of bacterial stress at 35° C., 37° C. and 39° C. The antibacterial activity of Nod2 increased with bacterial stress.

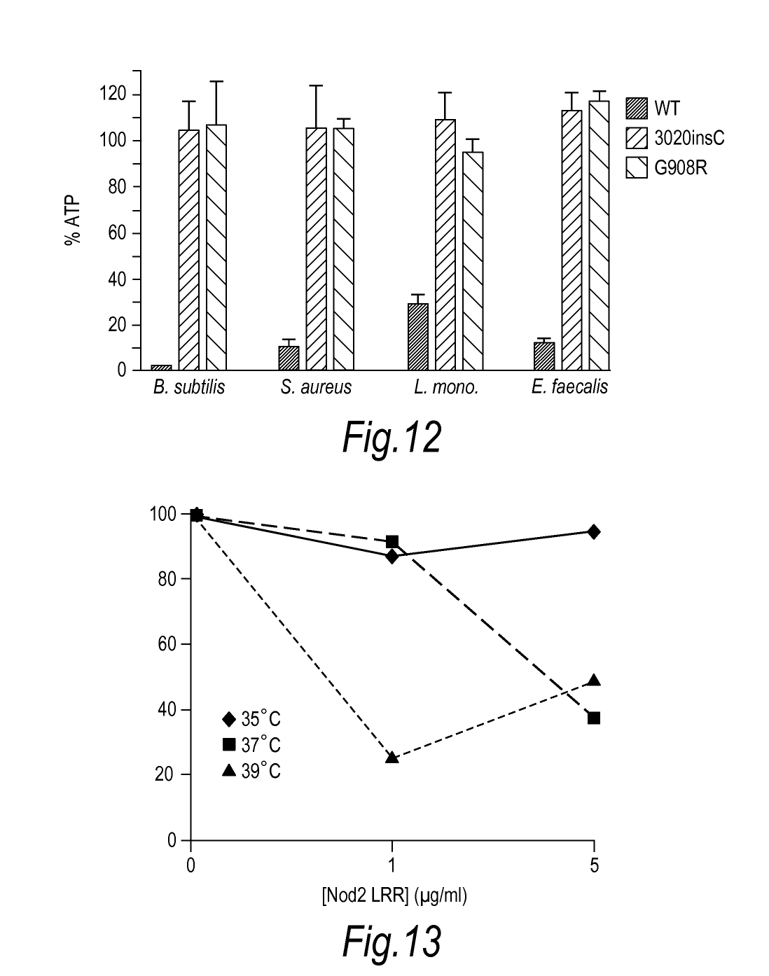

FIG. 14: Increasing concentrations of Nod2 inhibits the growth of B. Subtilis (Panel A) and S. aureus (Panel B). Values shown are relative to controls incubated in the absence of LRR domains (100%).

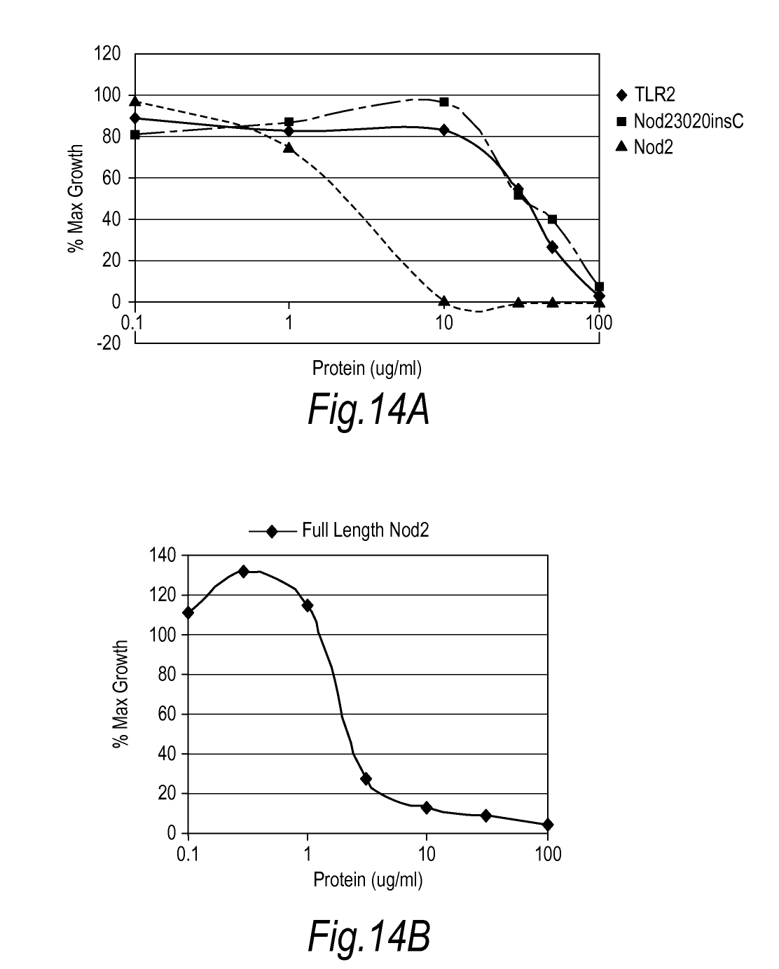

FIG. 15: NAIP inhibited the growth of S. maltophilia (Panel A). Nod1 inhibited E. coli growth (Panel B) and L. monocytogenes growth was inhibited by Nod1, Nod2, Nod2 3020insC and CIAS1 (Panel C).

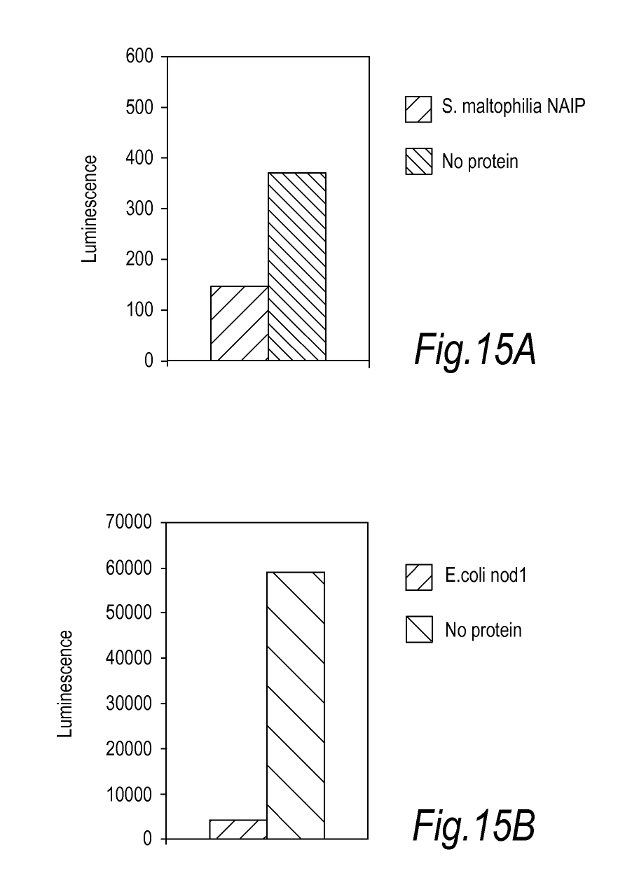

FIG. 16: Nod2 inhibited the growth of S. aureus. This inhibition was unaffected by the co-administration of MDP, LPS, or PGN.

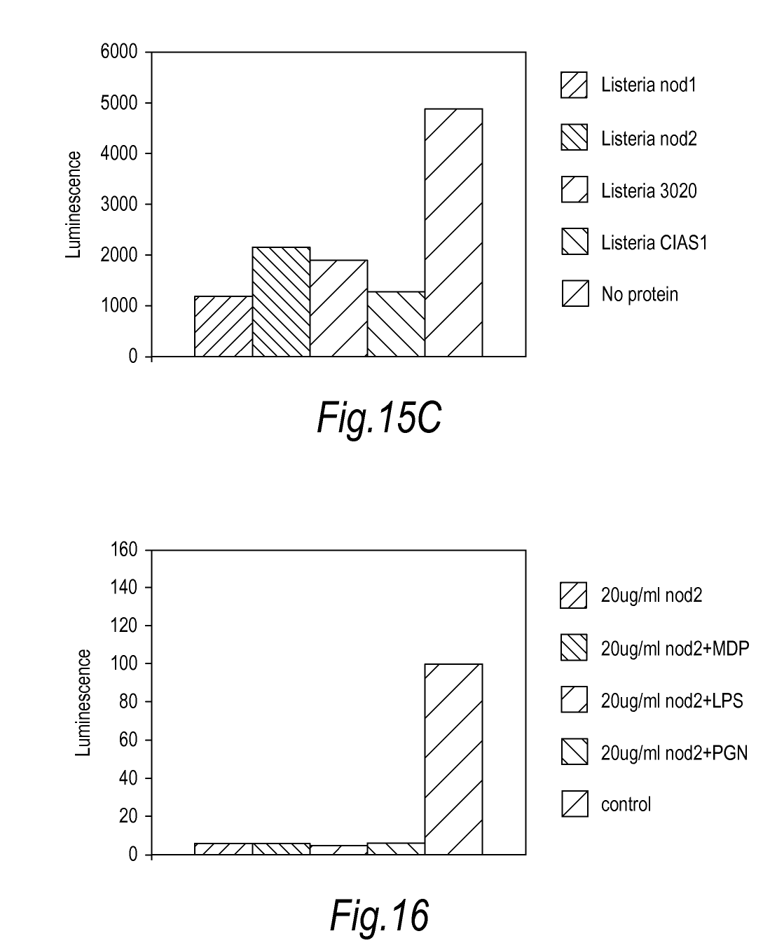

### 5. DETAILED DESCRIPTION OF THE INVENTION

In accordance with the present invention there is hence provided isolated proteins comprising a plurality of LRR (leucine rich repeat) domains, for use as an antimicrobial agent. In use of the invention as set out for example below it has been found that these proteins are effective in killing a wide range of bacteria and at potencies comparable to known antibiotics.

In preferred embodiments of the invention there is an LRR at the C-terminus of the protein. This has been found to increase the antimicrobial activity of the proteins.

It is further preferred that each LRR domain independently comprises or consists essentially of an amino acid sequence of formula (I):

(F1LxxLxL(xxZ)_{Y}F2) (I)
wherein:
F1 and F2 are independently, a contiguous amino acid sequence of between 1 and 30 residues;
x can be any amino acid;
L can be Leu, Ile, Val or Phe;
Z can be NxL or CxxL;
N is Asn, Thr, Ser or Cys;
C is Cys or Ser; and
Y=0 or 1.

Leucine rich repeats (LRRs) are generally protein structural motifs that form α/β horseshoe folds. Each LRR is typically composed of repeating 20-30 amino acid stretches that are unusually rich in leucine residues, though these can be substituted by other hydrophobic residues. Each repeat unit can have beta strand-turn-alpha helix structure, such that an assembled section, composed of a plurality of such LRRs, has a horseshoe or arc shape with an interior parallel beta sheet and an exterior array of helices. One face of the beta sheet and one side of the helix array are exposed to solvent and are therefore typically dominated by hydrophilic residues. The region between the helices and sheets generally forms a hydrophobic core, typically being tightly sterically packed with leucine residues. In alternative embodiments of the invention, other hydrophobic amino acid residues such as isoleucine, valine, phenylalanine, methionine, tryptophan or cysteine can substitute the leucine residues.

Generally, in the proteins of the invention all of the LRR domains form a single continuous structure and adopt an arc or horseshoe shape. The inner, concave face of the arc or horseshoe can be predominantly comprised of parallel β-strands, while the outer, convex face may comprise a number of secondary structures such as α-helix, 3₁₀-helix, polyproline II helix, or a tandem arrangement of β-turns. In embodiments of the invention the β-strands on the concave face and the mainly helical elements of the convex face are connected by short loops or β-turns.

Proteins of the invention comprise sufficient LRRs to have antimicrobial activity, and proteins of the invention suitably comprise from 3 to 20 LRR domains. Particular embodiments of the invention comprise at least 3 LRRs, at least 5 LRRS or at least 7 LRRs. In other embodiments of the invention the proteins can comprise at least 4, at least 6, at least 8, at least 9, at least 10, at least 11, at least 12, at least 13, at least 14, at least 15, at least 16, at least 17, at least 18, at least 19 or at least 20 LRR domains.

In a class of proteins which form an embodiment of the invention there is a high proportion of leucine residues present. Thus, at least 2 L residues in each LRR are Leu, or at least 3 L residues in each LRR are Leu. In certain embodiments substantially all L residues are Leu. Proteins of the invention are further preferably water soluble.

A particular sub-class of proteins of the invention comprise 5 or more LRR (leucine rich repeat) domains, for use as an antibacterial agent, wherein the C-terminus of the protein is an LRR domain and each LRR domain comprises an amino acid sequence of formula (I):

(F1LxxLxL(xxZ)_{Y}F2) (I)
wherein:
F1 and F2 are independently, a contiguous amino acid sequence of between 1 and 30 residues;
x can be any amino acid;
L can be Leu, Ile, Val or Phe;
Z can be NxL or CxxL;
N is Asn, Thr, Ser or Cys;
C is Cys or Ser; and
Y=0 or 1.

In this sub-class of proteins, at least 2 L residues in each LRR are preferably Leu.

The term “isolated” as used herein refers to proteins and polynucleotides of the invention, as the case maybe, that exist in a physical milieu distinct from that in which it occurs in nature. For example, the isolated protein or polynucleotide may be substantially isolated (for example purified) with respect to the complex cellular milieu in which it naturally occurs. It should be noted however that although a protein of the invention maybe described herein as “isolated” this does not imply that the protein must exist in nature.

The term “derived from” and “is derived” refers to the protein or polynucleotide in question regardless of its physical origin. Therefore, by way of example, “LRRs (e.g. in tandem) of formula (I) derived from a naturally occurring LRR containing protein” refers to LRRs that have the same primary amino acid sequence as found in the naturally occurring LRR containing protein but is not necessarily purified from that naturally occurring source.

The term “death fold domain” refers to a family of domains characterized by six tightly packed a helices that play a prominent role in programmed cell death (apoptosis). Members of this family include caspase recruitment domain (CARD), pyrin domain (PYD), death domain (DD) and death effector domain (DED). The reader is specifically referred to Lahm A et al (2003); Cell death and Differentiation, 10, 10-12 and references cited therein for further information on this family.

The term “LRR” or “LRR motif” and grammatical variations thereof refers to a leucine rich repeat motif of formula (I).

The term “LRR domain” refers to a protein domain comprising (or consisting essentially of, or consisting of) two or more (up to about fifty), typically in tandem, LRRs of formula (I).

The term “NOD protein” refers to proteins that contain a central nucleotide-binding oligomerization domain (NOD), an amino terminal CARD domain and a carboxyl terminal LRR domain. The reader is specifically referred to Table I, page 361 of Inohara N. et al (2005), Annu. Rev. Biochem 74:355-383 for details of (not necessarily exhaustive) members of the human NOD family.

The suffix “-LRR” refers to the naturally occurring LRR domain of the preceding protein and therefore “NOD-LRR” refers to the LRR domain found in naturally occurring members of the NOD family.

“LRR protein” means a protein comprising at least one LRR domain.

“TLR” refers to the toll like receptor family. Toll-like receptors (TLRs) are a class of single membrane-spanning non-catalytic receptors that recognize structurally conserved molecules derived from microbes. See Mitchell J A (2007), J Endocrinol 193(3); 323-30 the entire contents of which are incorporated by reference and to which the reader is specifically referred.

“Protein” includes polypeptide.

“human NOD2” refers to the protein of SEQ ID NO: 1.

“human NOD1” refers to the protein of SEQ ID NO: 2.

“human NOD2-LRR” refers to the protein of SEQ ID NO: 3

“human NOD1-LRR” refers to the protein of SEQ ID NO: 4.

“human CIITA-LRR” refers to the protein of SEQ ID NO: 5

“human TLR2-LRR” refers to the protein of SEQ ID NO: 6.

“human Nalp3-LRR” refers to the protein of SEQ ID NO: 7.

“anti-bacterial pharmaceutical composition” refers to a pharmaceutical composition that possesses anti-bacterial activity, inter alia, before administration into a subject.

### 5.1 Proteins

The present invention is based, at least in part, on the surprising observation that proteins containing an LRR motif (of formula (I)) have anti-bacterial (particularly bactericidal) activity. Although we demonstrate that naturally occurring proteins comprising LRR domains together with other domains (such as seen in the NOD family of proteins) have significant anti-bacterial activity, we also demonstrate that LRR domains, by themselves, possess anti-bacterial activity.

In some embodiments, the isolated protein comprises between 2 and 100 tandemly arranged LRR motifs of formula (I), more particularly between 2 and 50, e.g. between 2 and 45.

In typical embodiments, the LRR motif is between 15 and 50 residues long e.g. 20 to 30 residues long. Therefore in some embodiments, the isolated protein comprises between two and one hundred tandemly arranged LRR motifs of formula (I) (for example between two and fifty) each motif consisting of between 15 and 50 contiguous amino acid residues (e.g. 20 to 30 residues).

In some embodiments, the protein is artificial, that is it has an arrangement not found in nature. In these embodiments, the protein may comprise a central nucleotide-binding oligomerization domain (NOD), a carboxyl terminal LRR domain (comprising e.g. an artificial number of LRR domains, preferably arranged in tandem) and an amino terminal effector domain. The effector domain may, for example, promote killing (e.g. by apoptosis) of a target cell such as a pathogenic bacteria. Examples of such effector domains are the death fold domains such as CARD, Pyrin, Death Domain and Death effector domain.

In other aspects of the invention, there is provided an isolated protein comprising an LRR domain derived from a naturally occurring protein. In some embodiments of this aspect of the invention, the isolated protein is a naturally occurring protein comprising a LRR domain (sometimes referred to herein as an “LRR protein”). The naturally occurring LRR protein maybe “RI-like”, “CC”, “bacterial”, “SDS22-like”, “plant specific”, “typical” or “TpLRR”, see Kajava A. V. (1998), J. Mol. Biol. 277, 519-527 and Ohyanagi T et al (1997), FASEB J 11:A949, both of which are incorporated herein in their entirety and to which the reader is specifically referred. Examples of such naturally occurring proteins are animal derived proteins and include members of the NOD family, and in particular human (or other primate) NOD proteins (such as human NOD1 or human NOD2). Other members include the Toll-like receptors (TLR) family and include TLR 2,4,5,7,8 and 9 and in particular human and other mammalian orthologues thereof. Other further examples include members include CIITA and NAIP.

In some embodiments, the isolated protein is selected from the group consisting of; SEQ ID NO: 1, 2, 3, or 4.

In other aspects of the invention, there is provided isolated LRR domains, that is a protein that consists of an isolated LRR domain. In some embodiments, the protein maybe an isolated LRR domain

In other aspects of the invention there is provided an isolated LRR protein with the proviso that the LRR protein is not an isolated polypeptide comprising an N-terminal leucine rich repeat, one or more leucine rich repeats, a C-terminal leucine rich repeat, and a connecting peptide wherein the connecting peptide comprises an alpha helix.

### 5.2 Polynucleotides.

In other aspects of the invention there is provided isolated polynucleotides (such as RNA or cDNA) that encode proteins of the invention. Such polynucleotides may be used in processes for the manufacture of isolated proteins of the invention, for example in the manufacture of a medicament (such as a pharmaceutical composition) comprising an isolated protein of the invention. In other aspects, polynucleotides encoding proteins of the invention maybe incorporated into a vector such as a plasmid, virus, minichromosome, transposon and the like as part of a therapeutic or prophylactic immunogenic composition (such as a vaccine, e.g. a DNA vaccine) to augment host defence against pathogens such as pathogenic bacteria.

Therefore in one aspect of the invention there is provided an isolated polynucleotide such as DNA (e.g. cDNA) or RNA that encodes a protein comprising (or consisting essentially of or consisting of) an LRR of formula (I).

In another aspect of the invention there is provided an isolated polynucleotide such as DNA (e.g. cDNA) or RNA that encodes a protein comprising a LRR domain. In some embodiments of this aspect there is provided an isolated polynucleotide that encodes a naturally occurring LRR domain such as a NOD LRR domain, particularly a human NOD LRR domain such as a protein of SEQ ID NO: 2 or 3.

In another aspect of the invention there is provided an isolated polynucleotide such as DNA (e.g. cDNA) or RNA that encodes a LRR protein, in particular an animal derived naturally occurring LRR protein such as a NOD protein and more particularly a human or other primate NOD protein. Examples therewith include isolated polynucleotides that encode human NOD1 or human NOD2. Other examples include isolated polynucleotides that encode Toll like receptor (TLR) for example, TLR2,7, 8 or 9 and CIITA or NAIP.

### 5.3 Production Processes

Certain aspects of the invention concern processes for producing isolated proteins and proteins of the invention and in particular those mentioned in section 5.1.

Isolated proteins and proteins of the invention are typically produced using recombinant cell culturing technology well known to those skilled in the art. A polynucleotide encoding the protein or protein is isolated and inserted into a replicable vector such as a plasmid for further cloning (amplification) or expression. One useful expression system is a glutamate synthetase system (such as sold by Lonza Biologies), particularly where the host cell is CHO or NSO (see below). Polynucleotide encoding the polynucleotide or protein is readily isolated and sequenced using conventional procedures (e.g. oligonucleotide probes). Vectors that may be used include plasmid, virus, phage, transposons, minichromsomes of which plasmids are a typical embodiment. Generally such vectors further include a signal sequence, origin of replication, one or more marker genes, an enhancer element, a promoter and transcription termination sequences operably linked to the polynucleotide so as to facilitate expression.

### 5.3.1 Signal Sequences

Proteins of the present invention maybe produced as a fusion protein with a heterologous signal sequence having a specific cleavage site at the N terminus of the mature protein. The signal sequence should be recognised and processed by the host cell. For prokaryotic host cells, the signal sequence may be an alkaline phosphatase, penicillinase, or heat stable enterotoxin Il leaders. For yeast secretion the signal sequences may be a yeast invertase leader, [alpha] factor leader or acid phosphatase leaders see e.g. WO90/13646. In mammalian cell systems, viral secretory leaders such as herpes simplex gD signal and a native immunoglobulin signal sequence are available. Typically the signal sequence is ligated in reading frame to DNA encoding the antibody of the invention.

### 5.3.2 Origin of Replication

Origin of replications are well known in the art with pBR322 suitable for most gram-negative bacteria, 2μ plasmid for most yeast and various viral origins such as SV40, polyoma, adenovirus, VSV or BPV for most mammalian cells. Generally the origin of replication component is not needed for mammalian expression vectors but the SV40 may be used since it contains the early promoter.

### 5.3.3 Selection Marker

Typical selection genes encode proteins that (a) confer resistance to antibiotics or other toxins e.g. ampicillin, neomycin, methotrexate or tetracycline or (b) complement auxotrophic deficiencies or supply nutrients not available in the complex media. The selection scheme may involve arresting growth of the host cell. Cells, which have been successfully transformed with the genes encoding the therapeutic antibody of the present invention, survive due to e.g. drug resistance conferred by the selection marker. Another example is the so-called DHFR selection marker wherein transformants are cultured in the presence of methotrexate. In typical embodiments, cells are cultured in the presence of increasing amounts of methotrexate to amplify the copy number of the exogenous gene of interest. CHO cells are a particularly useful cell line for the DHFR selection. A further example is the glutamate synthetase expression system (Lonza Biologies). A suitable selection gene for use in yeast is the trp1 gene, see Stinchcomb et al Nature 282, 38, 1979.

### 5.3.4 Promoters

Suitable promoters for expressing proteins and polynucleotides of the invention are operably linked to DNA/polynucleotide encoding the antibody. Promoters for prokaryotic hosts include phoA promoter, Beta-lactamase and lactose promoter systems, alkaline phosphatase, tryptophan and hybrid promoters such as Tac. Promoters suitable for expression in yeast cells include 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase. Inducible yeast promoters include alcohol dehydrogenase 2, isocytochrome C, acid phosphatase, metallothionein and enzymes responsible for nitrogen metabolism or maltose/galactose utilization.

Promoters for expression in mammalian cell systems include viral promoters such as polyoma, fowlpox and adenoviruses (e.g. adenovirus 2), bovine papilloma virus, avian sarcoma virus, cytomegalovirus (in particular the immediate early gene promoter), retrovirus, hepatitis B virus, actin, rous sarcoma virus (RSV) promoter and the early or late Simian virus 40. Of course the choice of promoter is based upon suitable compatibility with the host cell used for expression. In one embodiment therefore there is provided a first plasmid comprising a RSV and/or SV40 and/or CMV promoter, DNA encoding light chain V region (VL) of the invention, KC region together with neomycin and ampicillin resistance selection markers and a second plasmid comprising a RSV or SV40 promoter, DNA encoding the heavy chain V region (VH) of the invention, DNA encoding the [gamma] 1 constant region, DHFR and ampicillin resistance markers

### 5.3.5 Enhancer Element

Where appropriate, e.g. for expression in higher eukaroytics, an enhancer element operably linked to the promoter element in a vector may be used. Suitable mammalian enhancer sequences include enhancer elements from globin, elastase, albumin, fetoprotein and insulin. Alternatively, one may use an enhancer element from a eukaroytic cell virus such as SV40 enhancer (at bp100-270), cytomegalovirus early promoter enhancer, polyma enhancer, baculoviral enhancer or murine IgG2a locus (see WO04/009823). The enhancer is preferably located on the vector at a site upstream to the promoter.

### 5.3.6 Host Cells

Suitable host cells for cloning or expressing vectors encoding isolated proteins of the invention are prokaroytic, yeast or higher eukaryotic cells. Suitable prokaryotic cells include eubacteria e.g. enterobacteriaceae such as Escherichia e.g. E. Coli (for example ATCC 31, 446; 31, 537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. licheniformis (see DD 266 710), Pseudomonas such as P. aeruginosa and Streptomyces. Of the yeast host cells, Saccharomyces cerevisiae, schizosaccharomyces pombe, Kluyveromyces (e.g. ATCC 16,045; 12,424; 24178; 56,500), yarrowia (EP402, 226), Pichia Pastoris (EP183, 070, see also Peng et al J. Biotechnol. 108 (2004) 185-192), Candida, Thchoderma reesia (EP244, 234J, Penicillin, Tolypocladium and Aspergillus hosts such as A. nidulans and A. niger are also contemplated.

Host cells of the present invention maybe higher eukaryotic cells. Suitable higher eukaryotic host cells include mammalian cells such as COS-1 (ATCC No. CRL 1650) COS-7 (ATCC CRL 1651), human embryonic kidney line 293, baby hamster kidney cells (BHK) (ATCC CRL.1632), BHK570 (ATCC NO: CRL 10314), 293 (ATCC NO. CRL 1573), Chinese hamster ovary cells CHO (e.g. CHO-K¹, ATCC NO: CCL 61, DHFR-CHO cell line such as DG44 (see Urlaub et al, (1986) Somatic Cell Mol. Genet. 12, 555-556)), particularly those CHO cell lines adapted for suspension culture, mouse Sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NSO (see U.S. Pat. No. 5,807,715), Sp2/0, YO. Thus in one embodiment of the invention there is provided a stably transformed host cell comprising a vector encoding an isolated protein comprising two or more LRRs of formula (I), a LRR domain or a LRR protein.

### 5.3.7 Bacterial Fermentation

Bacterial systems maybe used to produce proteins of the invention. Typically they are produced as insoluble periplasmic proteins which can be extracted and refolded to form active proteins according to methods known to those skilled in the art, see Sanchez et al (1999) J. Biotechnol. 72, 13-20 and Cupit P M et al (1999) Lett Appl Microbiol, 29, 273-277.

### 5.3.8 Cell Culturing Methods.

Host cells transformed with vectors encoding the proteins of the invention or antigen binding fragments thereof may be cultured by any method known to those skilled in the art. Host cells may be cultured in spinner flasks, roller bottles or hollow fibre systems but it is preferred for large scale production that stirred tank reactors are used particularly for suspension cultures. Preferably the stirred tankers are adapted for aeration using e.g. spargers, baffles or low shear impellers. For bubble columns and airlift reactors direct aeration with air or oxygen bubbles maybe used. Where the host cells are cultured in a serum free culture media it is preferred that the media is supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly invertebrate host cells may utilise a variety of operational modes such as fed-batch, repeated batch processing (see Drapeau et al (1994) cytotechnology 15: 103-109), extended batch process or perfusion culture. Although recombinantly transformed mammalian host cells may be cultured in serum-containing media such as fetal calf serum (FCS), it is preferred that such host cells are cultured in synthetic serum-free media such as disclosed in Keen et at (1995) Cytotechnology 17:153-163, or commercially available media such as ProCHO-CDM or UltraCHO™ (Cambrex N.J., USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg K et al (1995) in Animal Cell technology: Developments towards the 21st century (Beuvery E. G. et al eds), pp 619-623, Kluwer Academic publishers).

Proteins of the invention secreted into the media may be recovered and purified using a variety of techniques to provide a degree of purification suitable for the intended use. For example the use of therapeutic proteins of the invention for the treatment of human patients typically mandates at least 95% purity, more typically 98% or 99% or greater purity (compared to the crude culture medium). In the first instance, cell debris from the culture media is typically removed using centrifugation followed by a clarification step of the supernatant using e.g. microfiltration, ultrafiltration and/or depth filtration. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. Typically, various virus removal steps are also employed (e.g. nanofiltration using e.g. a DV-20 filter). Following these various steps, a purified preparation comprising at least 35 mg/ml or greater e.g. 100 mg/ml or greater of the isolated protein of the invention thereof is provided and therefore forms an embodiment of the invention. Suitably such preparations are substantially free of aggregated forms of proteins of the invention.

### 5.4. Pharmaceutical Compositions

In certain embodiments, isolated proteins and polynucleotides of the invention are incorporated into a pharmaceutical composition for treating and/or preventing pathogenic bacteria infection. In some embodiments, the pharmaceutical composition is for treating and/or preventing infection by bacteria pathogenic to humans. In other embodiments, the pharmaceutical composition is for treating and/or preventing bacterial infection pathogenic to non-human animals e.g. for veterinarian use. Embodiments for treating and/or preventing infection by specific pathogenic bacteria is noted in more detail below. The reader may assume that it is intended that each and every protein or polynucleotide aspect or embodiment set forth in section 3, section 5.1 and section 5.2 are specifically and individually contemplated herein to be incorporated into a pharmaceutical composition.

In general, pharmaceutical compositions of the invention comprise (or consist essentially of) a therapeutically effective amount (for example in unit dosage amount) of an isolated protein of the invention together with a pharmaceutically acceptable carrier as known and called for by accepted pharmaceutical practice. The formulation of proteins for pharmaceutical use is well understood and the reader is referred in particular to Hovgaard L (2000) “Pharmaceutical formulation development of peptides and proteins”, CRC Press, ISBN: 0748407456; Nail S. et al (2002) “Development and manufacture of protein pharmaceuticals”, Springer, ISBN: 0306467453; McNally E. J. (1999) “Protein formulation and delivery (Drugs & the Pharmaceutical Sciences), Marcel Dekker Ltd, ISBN: 0824778839. See also Remington's Pharmaceutical Sciences, 16th ed., 1980, Mack Publishing Co., edited by Oslo et al. the disclosure of which is hereby incorporated by reference. Pharmaceutical compositions of the invention may be rendered suitable for administration by any convenient or necessary route depending on the underlying disease or condition it is desired to treat. Thus in some embodiments there is provide an intravenously administratable pharmaceutical composition comprising a therapeutically effective amount of a protein of the invention. In other embodiments, there is provide a pharmaceutical composition suitable for sub-cutaneous administration of a therapeutically effective amount of a protein of the invention.

The protein of the invention is prepared for storage or administration by mixing protein of the invention having the desired degree of purity with physiologically acceptable carriers, excipients, or stabilizers. Such materials are non-toxic to recipients at the dosages and concentrations employed. If the protein of the invention is water soluble, it may be formulated in a buffer such as phosphate or other organic acid salt preferably at a pH of about 7 to 8. If protein is only partially soluble in water, it may be prepared as a microemulsion by formulating it with a nonionic surfactant such as Tween, Pluronics, or PEG, e.g., Tween 80, in an amount of 0.04-0.05% (w/v), to increase its solubility.

Optionally other ingredients may be added such as antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The protein of the invention to be used for therapeutic administration must be sterile. Sterility is readily accomplished by filtration through sterile filtration membranes (e.g., 0.2 micron membranes). The protein of the invention ordinarily will be stored in lyophilized form or as an aqueous solution if it is highly stable to thermal and oxidative denaturation. The pH of the protein preparations of the invention typically will be about from 6 to 8, although higher or lower pH values may also be appropriate in certain instances. It will be understood that use of certain of the foregoing excipients, carriers, or stabilizers will result in the formation of salts of the proteins of the invention.

If the protein of the invention is to be used parenterally, therapeutic compositions containing the protein of the invention generally are placed into a container having a sterile access port, for example, an intravenous solution bag or vial having a stopper pierceable by a hypodermic injection needle.

Generally, where the disease/disorder permits, one should formulate and dose the protein of the invention for site-specific delivery. This is convenient in the case of wounds and ulcers. For example, the protein of the invention maybe incorporated into a gel (e.g. a hydrogel) and administered into the wound or ulcer bed.

Sustained release formulations may also be prepared, and include the formation of microcapsular particles and implantable articles. For preparing sustained-release compositions, the protein of the invention is preferably incorporated into a biodegradable matrix or microcapsule. A suitable material for this purpose is a polylactide, although other polymers of poly-(α-hydroxycarboxylic acids), such as poly-D-(−)-3-hydroxybutyric acid (EP 133,988A), can be used. Other biodegradable polymers include poly(lactones), poly(acetals), poly(orthoesters), or poly(orthocarbonates). The initial consideration here must be that the carrier itself, or its degradation products, is nontoxic in the target tissue and will not further aggravate the condition. This can be determined by routine screening in animal models of the target disorder or, if such models are unavailable, in normal animals. Numerous scientific publications document such animal models.

For examples of sustained release compositions, see U.S. Pat. No. 3,773,919, EP 58,481A, U.S. Pat. No. 3,887,699, EP 1 58,277A, Canadian Patent No. 1176565, U. Sidman et al., Biopolymers 22, 547 [1983], and R. Langer et al., Chem. Tech. 12, 98 [1982].

When applied topically, the protein of the invention is suitably combined with other ingredients, such as carriers and/or adjuvants. There are no limitations on the nature of such other ingredients, except that they must be pharmaceutically acceptable and efficacious for their intended administration, and cannot degrade the activity of the active ingredients of the composition. Examples of suitable vehicles include ointments, creams, gels, or suspensions, with or without purified collagen. The compositions also may be impregnated into transdermal patches, plasters, and bandages, preferably in liquid or semi-liquid form.

For obtaining a gel formulation, the protein of the invention is formulated in a liquid composition may be mixed with an effective amount of a water-soluble polysaccharide or synthetic polymer such as polyethylene glycol to form a gel of the proper viscosity to be applied topically. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch; agar; alginic acid and alginates; gum arabic; pullullan; agarose; carrageenan; dextrans; dextrins; fructans; inulin; mannans; xylans; arabinans; chitosans; glycogens; glucans; and synthetic biopolymers; as well as gums such as xanthan gum; guar gum; locust bean gum; gum arabic; tragacanth gum; and karaya gum; and derivatives and mixtures thereof. The preferred gelling agent herein is one that is inert to biological systems, nontoxic, simple to prepare, and not too runny or viscous, and will not destabilize the protein of the invention held within it.

Preferably the polysaccharide is an etherified cellulose derivative, more preferably one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. Most preferred herein is methylcellulose.

The polyethylene glycol useful for gelling is typically a mixture of low and high molecular weight polyethylene glycols to obtain the proper viscosity. For example, a mixture of a polyethylene glycol of molecular weight 400-600 with one of molecular weight 1500 would be effective for this purpose when mixed in the proper ratio to obtain a paste.

The term “water soluble” as applied to the polysaccharides and polyethylene glycols is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

If methylcellulose is employed in the gel, preferably it comprises about 2-5%, more preferably about 3%, of the gel and the protein of the invention is present in an amount of about 300-1000 mg per ml of gel.

The dosage to be employed is dependent upon the factors described above. As a general proposition, the protein of the invention is formulated and delivered to the target site or tissue at a dosage capable of establishing in the tissue a level greater than about 0.1 ng/cc up to a maximum dose that is efficacious but not unduly toxic. This intra-tissue concentration should be maintained if possible by continuous infusion, sustained release, topical application, or injection at empirically determined frequencies.

Compositions particularly well suited for the clinical administration of proteins of the invention hereof employed in the practice of the present invention include, for example, sterile aqueous solutions, or sterile hydratable powders such as lyophilized protein. It is generally desirable to include further in the formulation an appropriate amount of a pharmaceutically acceptable salt, generally in an amount sufficient to render the formulation isotonic. A pH regulator such as arginine base, and phosphoric acid, are also typically included in sufficient quantities to maintain an appropriate pH, generally from 5.5 to 7.5. Moreover, for improvement of shelf-life or stability of aqueous formulations, it may also be desirable to include further agents such as glycerol. In this manner, formulations are rendered appropriate for parenteral administration, and, in particular, intravenous administration.

Dosages and desired drug concentrations of pharmaceutical compositions of the present invention may vary depending on the particular use envisioned and are within the purview of the attending physician/healthcare professional.

In some embodiments therefore there is provided an anti-bacterial (e.g. bactericidal) pharmaceutical composition comprising an isolated NOD protein, particularly an isolated human NOD protein such as human NOD1 or human NOD2 (e.g. as its sole active ingredient).

In other embodiments there is provided a method of manufacturing a pharmaceutical composition, particularly an anti-bacterial pharmaceutical composition which method comprises providing an isolated NOD protein, particularly isolated human NOD protein such as human NOD1 and/or human NOD2.

In some other embodiments therefore there is provided an anti-bacterial (e.g. bactericidal) pharmaceutical composition comprising an isolated NOD-LRR domain, particularly an isolated human NOD-LRR domain such as human NOD1-LRR or human NOD2-LRR (e.g. as its sole active ingredient).

In some other embodiments therefore there is provided pharmaceutical composition (e.g. anti-bacterial such as bactericidal pharmaceutical composition) comprising as its sole active ingredient a protein consisting of an isolated LRR domain such as an isolated NOD-LRR domain particularly an isolated human NOD-LRR domain such as human NOD1-LRR or human NOD2-LRR (e.g. as its sole active ingredient) or an isolated human TLR-LRR domain such as TLR2-LRR, TLR4-LRR, TLR5-LRR, TLR9-LRR.

In other embodiments there is provided a method of manufacturing a pharmaceutical composition, particularly an anti-bacterial pharmaceutical composition which method comprises providing an isolated LRR protein such as an isolated human LRR protein such as an isolated NOD-LRR domain, particularly isolated human NOD-LRR domain such as human NOD1-LRR and/or human NOD2-LRR.

In other embodiments, there is provided an anti-bacterial (e.g. bactericidal) pharmaceutical composition comprising (for example as its sole active ingredient) an isolated TLR protein, for example an isolated mammalian TLR protein such as a human TLR 4, 5.

In other embodiments, there is provided an anti-bacterial (e.g. bactericidal) pharmaceutical composition comprising (for example as its sole active ingredient) an isolated TLR-LRR, for example, an isolated mammalian TLR-LRR such as human TLR4-LRR, human TLR5-LRR, human TLR2-LRR.

In other embodiments, there is provided a method of manufacturing an anti-bacterial (e.g. bactericidal) pharmaceutical composition which method comprises providing (for example as its sole active ingredient) an isolated TLR-LRR, for example, an isolated mammalian TLR-LRR such as human TLR4-LRR, human TLR5-LRR or human TLR2-LRR.

### 5.4.1 Other Compositions and Articles of Manufacture

In some embodiments, there is provided an effective amount of an isolated protein of the invention (such as detailed in sections 3 and 5.1 supra) incorporated into a disinfectant composition such as an aqueous disinfectant composition for disinfecting a surface or article in need thereof. The reader may assume that all aspects and embodiments set forth in sections 3 and 5.1 of this specification are individually and specifically contemplated to be of use in this section. Examples of such surfaces include those normally found in a clinical setting such as hospital wards, surgical surfaces and the like and other surfaces where it is desirable to reduce exposure to pathogenic bacteria. Disinfectant compositions of the invention may also be used to disinfect articles such as medical articles e.g. catheters or surgical instruments optionally in combination with other sterilization techniques as known and called for by good clinical practice. Proteins of the invention may also be used to disinfect water contaminated with pathogenic bacteria and the invention includes processes for disinfecting water contaminated with bacteria, particularly bacteria pathogenic to humans and/or other mammals which method comprises admixing said contaminated water with proteins of the invention.

In other embodiments, there is also provided a wound and/or surgical dressing comprising (or consisting essentially of) a protein of the invention.

### 5.5 Pathogenic Bacteria

In certain embodiments of the invention, compositions such as pharmaceutical compositions maybe used to treat and/or prevent infection by pathogenic bacteria. As noted, the bacteria maybe pathogenic to humans and/or other mammals. In some embodiments, the pathogenic bacteria is gram positive, in other embodiments, gram negative. In other contemplated embodiments the pathogenic bacteria are anaerobic bacteria pathogenic to the host (e.g. human). Examples of pathogenic bacteria include: Acinetobacter baumanii, Actinobacillis spp, Actinomycetes, Actinomyces (e.g. Actinomyces israelii, Actinomyces naeslundii, Actinomyces spp), Aeromonas spp (e.g. Aeromonas hydrophila, Aeromonas sobria, Aeromonas Caviae), Anaerobic Cocci such as Peptostreptococus, Veillonella, Gram positive Anaerobic Bacilli such as Mobiluncus spp, Propionibacterium acnes, Lactobacillus, Eubacterium, Bifidobacterium spp, Gram negative Anaerobic Bacilli such as Bacteroides, Prevotella spp, Porphyromonas spp, Fusobacterium spp, Bacillus spp (such as Bacillus anthracis, Bacillus cereus, Bacillus subtilis, Bacillus stearthermophilus), Bacteroides spp (such as Bacteroides fragilis), Bordetella spp (such as Bordetella pertussis, Bordetella parapertussis, Bordetella bronchiseptica), Borrelia spp (such as Borrelia recurrentis, Borrelia burgdorferi), Brucella spp (such as Brucella abortus, Brucella canis, Brucella melintensis, Brucella suis) Burkholderia spp (such as Burkholderia pseudomallei, Burkholderia cepacia), Campylobacter spp. (such as Campylobacter jejuni, Campylobacter coli, Campylobacter lari, Campylobacter fetus), Citrobacter spp (such as Citrobacter freundii, Citrobacter diversus), Clostridium spp (such as Clostridium perfingens, Clostridium difficile, Clostridium botulinum), Corynebacterium spp (such as Corynebacterium diphtheriae, Corynebacterium jeikeum, Corynebacterium urealyticum), Edwardsiella tarda, Enterobacter spp (such as Enterobacter aerogenes, Enterobacter agglomerans, Enterbacter cloacae), Escherichia coli (such as enterotoxigenic E. coli, enteroinvasive E. coli, enteropathogenic E. coli, enterohemorrhagic E. coli, uropathogenic E. coli), Klebsiella spp (such as Klebsiella pneumoniae, Klebsiella oxytoca), Morganella morganii, Proteus spp (such as Proteus mirabilis, Proteus vulgaris), Providencia spp (such as Providencia alcalifaciens, Providencia rettgeri, Providencia stuartii), Salmonella enterica (e.g Salmonella typhi, Salmonella paratyphi, Salmonella enteritidis, Salmonella cholerauis, Salmonella typhimurium) Serratia spp (Serratia marcesans, Serratia liquifaciens), shigella spp (such as Shigella dysenteriae, Shigella flexneri, Shigella boydii, Shigella sonnei), Yersinia spp (such as Yersinia enterocolitica, Yersinia pestis, Yersinia pseudotuberculosis), Enterococcus spp (such as Enterococcus faecalis, Enterococcus faecium), Erysipelothrix rhusopathiae, Francisella tularensis, Haemophilus spp (Haemophilus influenzae, Haemophilus dureyi, Haemophilus aegyptius, Haemophilus parainfluenzae, Haemophilus parahaemolyticus), Helicobacter spp (such as Helicobacter pylori, Helicobacter cinaedi, Helicobacter fennelliae), Legionella pneumophila, Leptospira interrogans, Listeria monocytogenes, Micrococcus spp, Moraxella catarrhalis, Mycobacterium leprae, Mycobacterium tuberculosis, Nocardia spp (such as Nocardia asteroides, Nocardia brasiliensis, Neisseria spp (such as Neisseria gonorrhoeae, Nesseria meningitides), Pasteurella multocida, Plesiomonas shigelloides, Pseudomonas aeruginosa, Rhodococcus spp, Staphylococcus spp (such as Staphylococcus aureus, particularly methicillin resistant Staphylococcus aureus (MRSA) and Vancomycin resistant Staphylococcus. aureus (VRSA), Staphylococcus epidermidis, Staphylococcus saprophyticus), Stenotrophomonas maltophilia, Streptococcus spp (such as Streptococcus pyogenes, Streptococcus agalactiae, Streptococcus anginosus, Streptococcus equismilis, Streptococcus bovis, Streptococcus anginosus, Streptococcus mutans, Streptococcus salivarius, Streptococcus sanguis, Streptococcus mitis, Streptococcus milleri), Streptomyces spp, Treponema spp (such as Treponema pallidum, Treponema endemicum, Treponema pertenue, Treponema carateum) Vibrio spp (such as Vibrio cholerae including pathogenic serotypes thereof such as O1 and O139, Vibrio parahaemolyticus, Vibrio vulnificus, Vibrio alginolyticus, Vibrio minicus, Vibrio fluvialis, Vibrio metchnikovii, Vibrio damsela, Vibrio furnisii).

Thus the present invention provides a pharmaceutical composition (and methods of treatment associated therewith) for treating and/or preventing infection by any one of the above named pathogenic bacteria, particularly in a human patient which composition comprises (or consists essentially of) any of the protein embodiments set forth in section 3 and/or section 5.1. The reader may assume that all possible combinations of proteins set forth in section 3 or section 5.1 are specifically and individually contemplated to be used to treat and/or prevent infection by any of the pathogenic bacteria set forth in this section and all such combinations each form a separate embodiment of the present invention. Specifically mentioned however are pharmaceutical compositions comprising or consisting essentially of a human LRR protein such as a human NOD protein (e.g. human NOD1 or human NOD2) or human TLR protein for the treatment and/or prevention of infection in humans by a pathogenic bacteria (for example a strain thereof) that is developing or has developed resistance to conventionally used drugs, e.g. MRSA and VRSA.

### 5.6 Clinical Diseases.

It will apparent to the skilled reader on the basis of the disclosure herein that compositions, particularly pharmaceutical compositions may be used to treat and/or prevent a number of diseases, particularly human diseases. Therefore pharmaceutical compositions comprising and/or consisting essentially of any of the proteins of section 3 and/or section 5.1 supra may be used to treat and/or prevent any one of the following infectious diseases, particularly in humans:

Anthrax, Bacterial Meningitis, Botulism, Brucellosis, Campylobacteriosis, Cat Scratch Disease, Cholera, Diphtheria, Epidemic Typhus, a food borne illness such as food poisoning, Gonorrhea, Impetigo, Legionellosis, Leprosy (Hansen's Disease), Leptospirosis, Listeriosis, Lyme disease, Melioidosis, MRSA infection, Meningitis, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus, Urinary Tract Infections.

In some embodiments, pharmaceutical compositions may be used to treat and/or prevent opportunistic infections in susceptible patients such as humans (e.g. in Cystic Fibrosis and/or humans that are immunosurpressed).

The reader may assume that all possible combinations of proteins of section 3 and section 5.1 supra are individually and specifically contemplated to be used in a composition such as a pharmaceutical composition to treat and/or prevent any one of the infectious diseases set forth supra.

In other embodiments, there is provided the use of a pharmaceutical composition comprising a protein as described in section 3 and 5.1 supra in treating and/or preventing diseases in which bacteria may play a pathological role. Examples thereof include peptic ulcer disease, and other gastrointestinal diseases such as Inflammatory bowel diseases (IBD) e.g. Crohns disease and Ulcerative Colitis, irritable bowel syndrome (IBS), and blood diseases such as sepsis.

In other embodiments there is provided the use of a pharmaceutical composition comprising a protein as described in section 3 and 5.1 in treating an inflammatory disease or disorder. Examples thereof include arthritic disorders such as psoriatic arthritis.

### 6. EXEMPLIFICATION

The present invention is described by way of the following non-limiting examples.

### 6.1 List of Abbreviations

| Abbreviation | Description |
|---|---|
| CIITA | class II, major histocompatibility complex, |
| | transactivator |
| GuHCl | Guanidinium hydrochloride |
| LRR | Leucine-rich repeat |
| MDP | Muramyldipeptide |
| MIC | Minimal inhibitory concentration |
| Naip | Neuronal apoptosis inhibitor protein |
| Nalp3 | Nacht Domain-, Leucine-Rich Repeat-, and |
| | PYD-Containing Protein 3 |
| NFkB | nuclear factor kappa-light-chain-enhancer of activated |
| | B cells |
| Nod1 | Nucleotide oligomerisation domain 1 |
| Nod2 | Nucleotide oligomerisation domain 2 |
| PFA | Paraformaldehyde |
| PRR | Pattern recognition receptor |
| SNP | Single nucleotide polymorphism |
| TLR2 | Toll-like receptor 2 |
| 3020insC | Crohn's-associated SNP of Nod2 |

### 6.2 Commercial Reagents

### 6.2.1 Bacteria

The following bacterial strains were purchased from the ATCC: Listeria monocytogenes (ATCC 7644), Bacillus subtilis (ATCC 6633), Enterococcus faecalis (ATCC 29212), Staphylococcus aureus (ATCC 29213), Streptococcus pneumoniae (ATCC 49619), Escherichia coli (ATCC 8739), Escherichia coli (ATCC 25922), Klebsiella pneumoniae (ATCC 700603), Pseudomonas aeruginosa (ATCC 27853), Salmonella choleraesuis (ATCC 13076), Stenotrophomonas maltophilia (ATCC 17666), Bacteroides fragilis (ATCC 25285), Fusobacterium nucleatum (ATCC 29148), Prevotella intermedia (clinical isolate), Eubacterium lentum (ATCC 43055), Clostridium perfringens (ATCC 13124), Clostridium difficile (clinical isolate), Clostridium ramosum (ATCC 25582), Peptostreptococcus anaerobius (ATCC 49031), Propionibacterium acnes (ATCC 25746).

### 6.2.2 Others

Proteoglycan, lipoteichoic acid, and heat killed Staphylococcus aureus were all purchased from Invivogen. Rhodamine-conjugated phalloidin was from Sigma.

### 6.2.3 Plasmids

A full-length NOD2 cDNA was obtained by assembling several PCR products from a peripheral blood lymphocyte library and cloned into the pENTR/SD/D-Topo vector (Invitrogen). A cDNA encoding NOD1 was purchased from Invitrogen (pENTR221-Nod1). The LRR domains of NOD1 and NOD2 were generated by PCR using primers flanking the LRR region, for NOD1: Nod1LRRFwd: 5′-caccatgaacaaggatcacttccagttcacc-3′ (SEQ ID NO: 8) and Nod1LRRrev: 5′-tcagaaacagataatccgcttctcatc-3′(SEQ ID NO: 9). For NOD2 Nod2LRRFwd: 5′-caccatgaccatgccagctgcaccgggtgagg-3′(SEQ ID NO: 10) and Nod2LRRrev: 5′-tcaaagcaagagtctggtgtccctgcagc-3′(SEQ ID NO: 11). To generate the Crohn's-associated 3020insC mutant of Nod2, a deoxycytosine was inserted at nucleotide position 3020 (NM_{—}022162). The integrity of all the cDNAs used was confirmed by DNA sequencing. The cDNAs encoding either full-length proteins or respective LRR domains were transferred into the following plasmids (Invitrogen) for the indicated applications: expression in 293 cells (pEF5/FRT/V5-Dest), bacterial expression (pDEST17), baculovirus assembly (pDEST10).

### 6.2.4 Antibodies

Commercial primary antibodies used were as follows: sheep, rabbit, and mouse secondary antibodies conjugated to Alexa-488, -568, or -647 were from Molecular Probes (Leiden, The Netherlands). IR-labeled secondary antibodies against rabbit or mouse were from Rockland Laboratories (West Grove, Pa.). Rabbit anti-NOD2 antibodies were generated by Eurogenetec using recombinant Nod2 LRR domains purified from E. coli as the immunogen. Serum was affinity purified using a recombinant LRR column. Specificity of the antibody was tested by western blot and immunofluorescence microscopy using cell lines expressing either recombinant Nod2 or Nod1.

6.3 293 Cell Lines Expressing Nod2, Nod2 3020insC, Nod1 and their Respective LRR Domains

Expression plasmids (pEF5/FRT/V5-DEST) containing cDNAs encoding Nod2, Nod2 3020insC, Nod1, Nod2 LRR, Nod2 3020insC LRR, Nod1 LRR were transfected into 293 Flp-In cells (Invitrogen) using Lipofectamine 2000 (Invitrogen) according to the manufacturer's recommendations. Stable cell lines were selected in 200 microgram/ml hygromycin. Expression of the respective proteins was confirmed by quantitative PCR and Western blotting.

### 6.3.1 Immunofluorescence

For immunostaining, intestinal epithelial cells were grown on glass coverslips and fixed in 3% paraformaldehyde (PFA) for 20 minutes. PFA-fixed cells were permeabilized with 0.1% Triton X-100 in PBS for 5 min. Antibody incubations were all carried out in PBS containing 0.2% BSA. Nuclei were stained with 0.5 mg/ml Hoechst (Sigma), and coverslips were mounted in pro-gold reagent (Invitrogen). Images were acquired on a Nikon eclipse microscope with standard objective lenses and filter sets. Images were processed with Adobe Photoshop 6.

### 6.3.2 Protein Purification

Complementary DNA sequences (as described in Section 6.2.3) encoding the LRR domains of NOD1, NOD2 and NOD2-3020 were transferred to the pDEST17 (Invitrogen) using the gateway technology by LR recombination. The LRR domains were overexpressed in Escherichia coli Rossetta (DE3) cells (Novagen), solubilized by guanidine-HCl (6M), spun at 15000×g and purified by sequential chromatography on Ni-NTA and HiLoad 16/60 Superdex 200 size-exclusion column. Purified protein were visualized by Coomassie blue staining The full-length NOD2 and NOD2-3020 cDNA were transferred from the pENTR/SD/D-Topo (Invitrogen) to the pDEST10 by LR recombination and them transformed in DH5αBac to produce the bacmid. Protocols from the Bac-to-Bac Baculovirus expression system (Invitrogen) were followed to obtain recombinant virus. For purification, of full length NOD2 and NOD2-3020insC, twenty T-162 Nunc tissue culture flasks with Hi5 cells were infected for 72 hr. Cells were scraped off and washed in cold PBS. The cell pellet was resuspended in 25 ml of 0.5M KCl, 50 mM tris, 10% glycerol, 5 mM mercaptoethanol, 1 mM MgCl2, 0.1% Triton X100, 10 mM imidazole and protease inhibitor (complete EDTA free (Roche)) (pH 7.0) and incubated for 15 min on ice. The suspension was sonicated twice for 40 sec, centrifuged 30 min, 15000×g at 40 C. The mixture was loaded sequentially onto Ni-NTA column and HiLoad 16/60 Superdex 200 size-exclusion columns. Purified protein were visualized by Coomassie blue staining

### 6.4 Antibacterial Assays

The BacTiter-Glo™ Microbial Cell Viability Assay (Promega) was used for determining the number of viable bacterial cells in culture based on quantitation of the ATP in individual cultures. Bacteria were inoculated at 5×105 cells/ml with the indicated concentration of proteins. The culture was incubated for 4 hrs at 37° C. and BacTiter-Glo reagent was directly added to bacterial cells in medium and luminescence was quantified using a Pherastar (BMG scientific). Values reported are the result of at least three individual experiments done in duplicate. Standard deviations for the IC50s were calculated using Excel (Microsoft). Values for the minimal inhibitory concentrations were determined with either aerobic or anaerobic cultures using standard procedures. Briefly, 5×10⁵ bacteria were inoculated into 0.1 ml of MHB broth containing the indicated concentration of LRR domain or antibiotic control. Cultures were incubated for 20-24 hours and the bacterial growth assessed by visual inspection with the aid of a viewing mirror. The MIC was determined as the lowest drug concentration that completely suppressed visual bacterial growth.

### 6.4.1 Gentamycin Protection Assay

Stable 293 FlpIn (Invitrogen) cell lines expressing chloramphenicol transferase (control), Nod2 or Nod2 3020insC were cultured on 24-well transwell culture plates (1×10⁵/well) (Corning Incorporated, Corning, N.Y.). After reaching confluence, Streptococcus pneumoniae was added at an MOI of 10:1. After 1-hour incubation at 37° C., cells were washed with Hanks' solution and cultured for 90 minutes in the presence of 0.5 mg/mL gentamycin/Hanks' solution to kill any extracellular bacteria. Cell lysates were then obtained by mechanical disruption and lysates diluted with 0.5 ml MHB broth and plated on chocolate agar plates. Plates were placed at 37° C. overnight and colonies were counted the following day.

### 6.4.2 Affinity Purification and Mass Spectrometry Protein Identification of Nod2 LRR-Associated Proteins

E. coli (ATCC 3556, ATCC 1655) were inoculated in MHB broth and the bacteria grown to saturation. The bacteria were harvested by centrifiguation (2800×g). Cells were lysed using an emulsiflex C5 and bacterial lysate separated by centrifugation for 30 minutes, 15000×g at 4° C. The bacterial pellet was recovered and resuspended in Triton X100 (1% in PBS) and spun again at 30000×g. The residual pellet was solubilized with guanidine-HCL (6M) and desalted by gel filtration. The proteins were refolded by rapid dilution and loaded on an activated NHS column covently linked to recombinant NOD2-LRR or NOD2-LRR-3020insC domains as indicated. Affinity-purified proteins were eluted from the column by salt gradient, separated by SDS-PAGE electrophoresis and analysed by Coomassie blue staining Identified protein bands were cut from the gel, reduced with DTT, alkylated with iodoacetamide and digested with modified trypsin at 37° C. overnight. The peptides were acidified with 1 microlitres of 100% formic acid prior analysis by LC-ESI-MS/MS. The nano-LC-MS experiments were performed using Eksigent/PAL HPLC system (Axel Semrau GmbH, Sprockhövel, Germany) connected to a LTQ-FT mass spectrometer (Thermo Electron, Bremen, Germany). The peptide mixtures were loaded directly to the analytical PicoFrit column (New Objective, Woburn, Mass.) and eluted from the column using a gradient from 98% phase A (0.1% formic acid aqueous solution) to 75% phase B (0.1% formic acid, 80% acetonitrile) in 50 min at 200 nl/min. The instrument was operated in a data-dependent acquisition mode automatically switching between MS and MS². The raw files were subsequently searched against the E. Coli sequence library using an in-house Mascot server (Matrix science Ltd., London, UK). The search was performed choosing trypsin as the enzyme with two miss cleavage allowed. Carboxymethyl (C) was chosen as the fixed modification and oxidation (M) as variable modification. The data were searched with a peptide mass tolerance of ±5 ppm and a fragment mass tolerance of ±0.8 Da. The proteins identified were accepted if at least two peptides were identified with a score above 20.

### 6.5 Results

Numerous independent studies have determined that specific SNPs in the LRR domain of Nod2 are a susceptibility factor for development of Crohn's disease. A robust immune response to commensal bacteria in the gastrointestinal tract is recognised as the major factor in the pathogenesis of the disease. The following experiments were performed to assess the functional role of Nod2 and the Crohn's-associated SNPs in the host response to bacteria.

### 6.5.1 In Vivo Expression of Nod2 in the Colon.

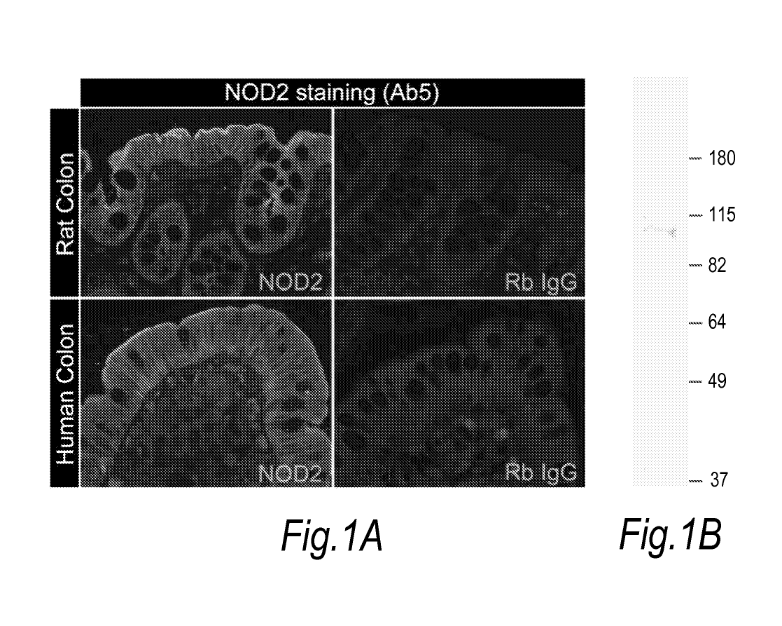

The gastrointestinal tract is home to approximately 10¹³ bacteria that are separated from their host by a single layer of epithelial cells. The expression of Nod2 protein in vivo was assessed using a polyclonal antibody against the LRR domain of Nod2 (FIG. 1). Immunohistochemical analysis determined that Nod2 was expressed primarily in the colonic epithelium. Intense staining was primarily found on the apical surface of the epithelium in direct contact with the commensal flora of the lumen. In addition, submucosal staining of macrophage and monocyte-like cells can be observed underlying the epithelium.

### 6.5.2 Cellular Nod2 Localization in Response to Bacteria

### Nod2 in Cultured SW480 Intestinal Epithelial Cells

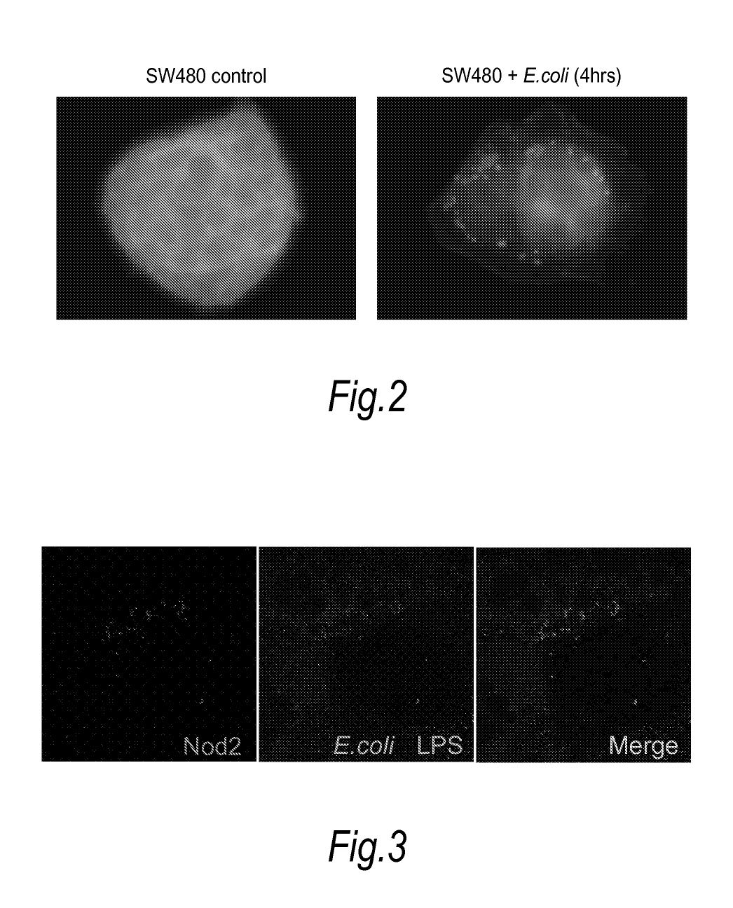

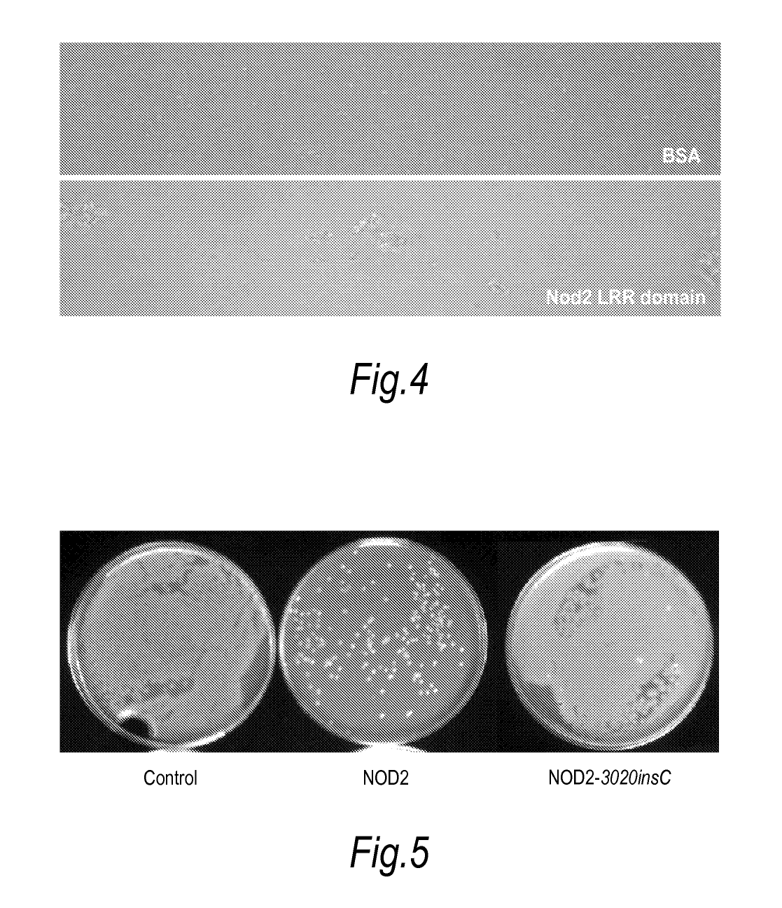

In order to investigate the function of Nod2 in the epithelium, SW480 intestinal epithelial cells were incubated with E. coli and the location of Nod2 in the cell determined by immunofluorescence (FIG. 2). In the absence of bacteria (SW480 control), Nod2 was found primarily in the cytosol of the cultured cells. Following incubation with E. coli, Nod2 could be observed in punctate, often oblong, structures approximately 1 micrometre in length within the cells. The observation of Nod2 in these distinct domains are reminiscent of the shape of E. coli itself, therefore additional experiments were performed to clarify the identification of Nod2 in these structures. The experiment was repeated with Caco2 intestinal epithelial cells. This cell line more closely expresses the phenotype of a normal epithelial layer in that they have tight junctions and develop trans-epithelial resistance. Incubation of these cells with E. coli resulted in Nod2 identification in similar punctate structures as were seen with SW480 cells following coculture with bacteria (FIG. 3). These cells were costained with an antibody specific for E. coli LPS, a component of the outer membrane of gram-negative bacteria. Clear colocalization was seen between LPS and Nod2 indicating that the Nod2-positive structures identified were bacteria.

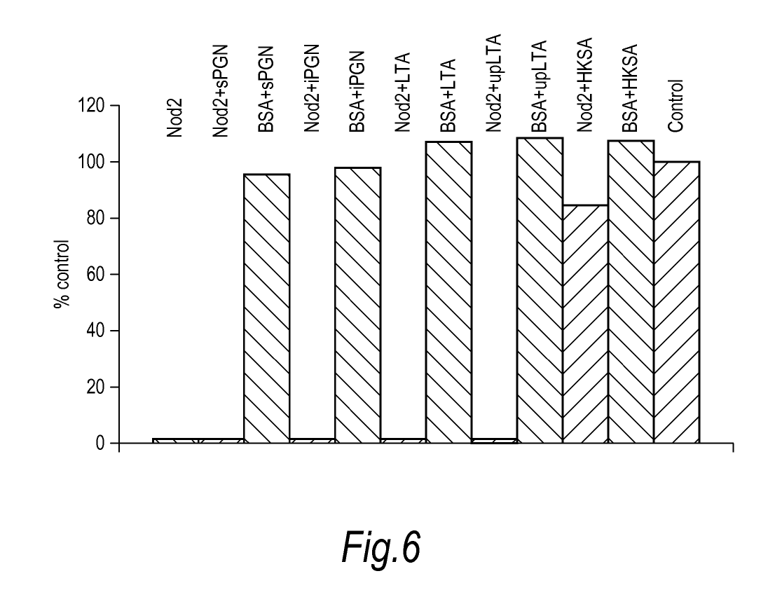

The Nod2 positive staining of the bacteria in the cytoplasm of cultured cells could either be a direct interaction or result from the colocalization of Nod2 and bacteria in an unidentified vesicular structure. In order to test the hypothesis that the interaction between Nod2 and E. coli was direct, purified recombinant LRR domains from Nod2 were incubated with E. coli (FIG. 4). Control cultures of E. coli in PBS demonstrated individual bacteria spread uniformly across the coverslip (top panel, FIG. 4). Following incubation with Nod2 LRR domains however, E. coli were aggregated and debris could be observed upon examination. This supports the hypothesis that Nod2 LRR domains can directly interact with E. coli.

### 6.5.3 Bacterial Infection

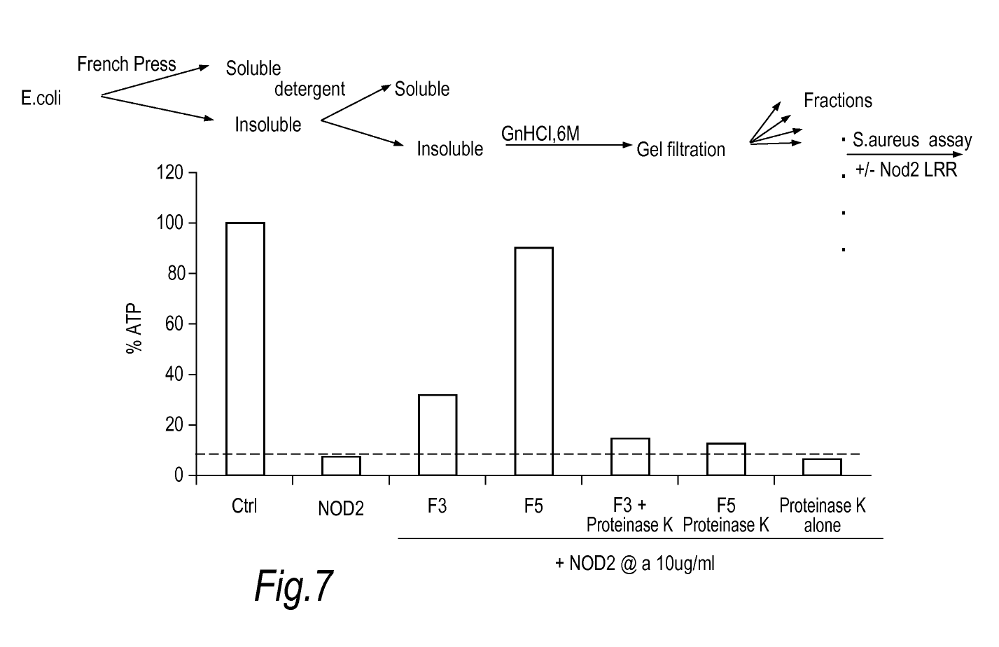

Previous studies have demonstrated a protective effect of Nod2 against infection by bacteria (Hisamatsu T, 2003). The data presented above suggest that this protective effect may be due to direct interaction of the Nod2 LRR domains to bacteria. In order to test this hypothesis, stable congenic cell lines expressing Nod2 or Nod2 3020insC LRR domains were constructed to control for protein expression levels and other factors. The cultured cells were inoculated with Streptococcus pneumoniae; a pathogen known to actively infect 293 cells in culture (Opitz B, 2004). The gentamycin-protected intracellular bacteria could then be assessed (FIG. 5). A lawn of bacteria were observed from the infected control cells. This number was drastically reduced in cells expressing the Nod2 LRR domain. No obvious protection from S. pneumoniae was observed in cells expressing the Crohn's-associated Nod2 3020insC LRR domain. This demonstrates that the signalling function of Nod2 is dispensable to protect cells from infection since the Card and Nacht domains were not expressed in these cell lines. Furthermore, the Nod2 LRR domain is sufficient to protect cells from bacterial infection.

### 6.5.4 Antibacterial Activity of Nod2 and LRR Domains In Vitro

The data presented demonstrate that Nod2 LRR domains directly bind to bacteria and protect cells from infection. Since the LRR domains do not have any capacity for signal transduction that has been reported, we investigated the hypothesis that Nod2 LRR domains are antimicrobial polypeptides. Increasing concentrations of purified recombinant LRR domains from Nod2, the Crohn's-associated Nod2 3020insC or Nod1 were incubated with a panel of aerobic gram-positive and gram-negative bacteria and the bacterial growth assessed by monitoring ATP concentration (Table 1). Antimicrobial activity could be demonstrated for the LRR domains. Several observations demonstrated specificity of Nod2 and Nod1 LRR domains for certain bacteria. Nod2 LRR domains were at least an order of magnitude more potent than Nod1 LRRs against E. faecalis and S. aureus. Nod1 LRR domains demonstrated a greater efficacy than Nod2 against some gram-negative bacteria such as E. coli (ATCC8739) and K. pneumoniae. In addition, Nod2 LRR domains were generally significantly more potent than Nod2 3020insC LRR domains against all sensitive bacteria, with the exception of L. monocytogenes. This demonstrates that the Crohn's-associated SNP is deficient in its antimicrobial activity and taken in the context of the current state of knowledge suggests that this is the fundamental cause of Crohn's in patients carrying this allele.

### 6.5.5 Aerobic Bacteria

TABLE 1
Nod LRR antibacterial activity against aerobic bacteria as determined by
bacterial ATP content (IC50).

| | | [LRR domain] (microgram/ml +/− SD, n = 3) | | |
|---|---|---|---|---|
| GRAM | Bacteria (ATCC) | Nod2 | Nod2 3020insC | Nod1 |
| + | Listeria monocytogenes (7644) | 13.7 +/− 13.0 | 16.5 +/− 13.3 | 32.0 +/− 2.1 |
| | Bacillus Subtilis (6633) | 3.9 +/− 0.6 | 54.0 +/− 20.2 | 15.5 +/− 12.0 |
| | Enterococcus faecalis (29212 | 6.8 +/− 1.6 | None detected | >100 |
| | Staphylococcus aureus (29213) | 6.0 +/− 4.0 | None detected | 111.3 +/− 13.0 |
| | Streptococcus pneumoniae (49619) | 3.0 +/− 1.6 | None detected | 13.5 +/− 4.9 |
| − | Escherichia coli (8739) | >100 | None detected | 29.0 +/− 2.8 |
| | Escherichia coli (25922) | >100 | None detected | >100 |
| | Klebsiella pneumoniae (700603) | None detected | None detected | 30.8 +/− 4.9 |
| | Pseudomonas aeruginosa (27853) | None detected | None detected | >100 |
| | Salmonella choleraesuis (13076) | None detected | None detected | >100 |
| | Stenotrophomonas maltophilia (17666) | None detected | None detected | >100 |

>100 indicates activity detected but <50% inhibition.

### 6.5.6 Anaerobic Bacteria

The vast majority of bacteria in the gastrointestinal tract are anaerobic. Therefore, the antimicrobial activity of Nod2 LRR domains was assessed against a panel of gram-positive and gram-negative anaerobic bacteria (Table 2). Ciprofloxacin is a broad-range antibiotic that is active against all of the strains tested. The activity of the recombinant Nod2 LRR domains against all the strains was comparable to ciprofloxacin on a weight (microgram/ml) basis. Importantly, when the molecular weight of the two compounds is considered, Nod2 LRR domains are approximately 25-200 times more potent than ciprofloxacin on a molar (mmoles/ml) basis against all the bacteria tested.

TABLE 2
Nod2 LRR minimal inhibitory concentration (MIC) against anaerobic
bacteria: comparison with ciprofloxacin (microgram/ml)

| | Strain # | NOD2 | Ciprofloxacin |
|---|---|---|---|
| Bacteroides fragilis | NB85001 | 4 | 8 |
| Fusobacterium nucleatum | NB86006 | 8 | 2 |
| Prevotella intermedia | NB88001 | 4 | 1 |
| Eubacterium lentum | NB94001 | 8 | 2 |
| Clostridium perfringens | NB95001 | 4 | 2 |
| Clostridium difficile | NB95002 | 4 | 8 |
| Clostridium ramosum | NB95010 | 4 | 8 |
| Peptostreptococcus anaerobius | NB97001 | 2 | 1 |
| Propionibacterium acnes | NB99001 | 4 | 1 |

Molecular weight of Nod2 LRR domain ~30000 da.
Molecular weight of Ciprofloxacin HCl = 386 da.

### 6.5.7 Other LRR Domains

### Antibacterial Mechanism of Nod2

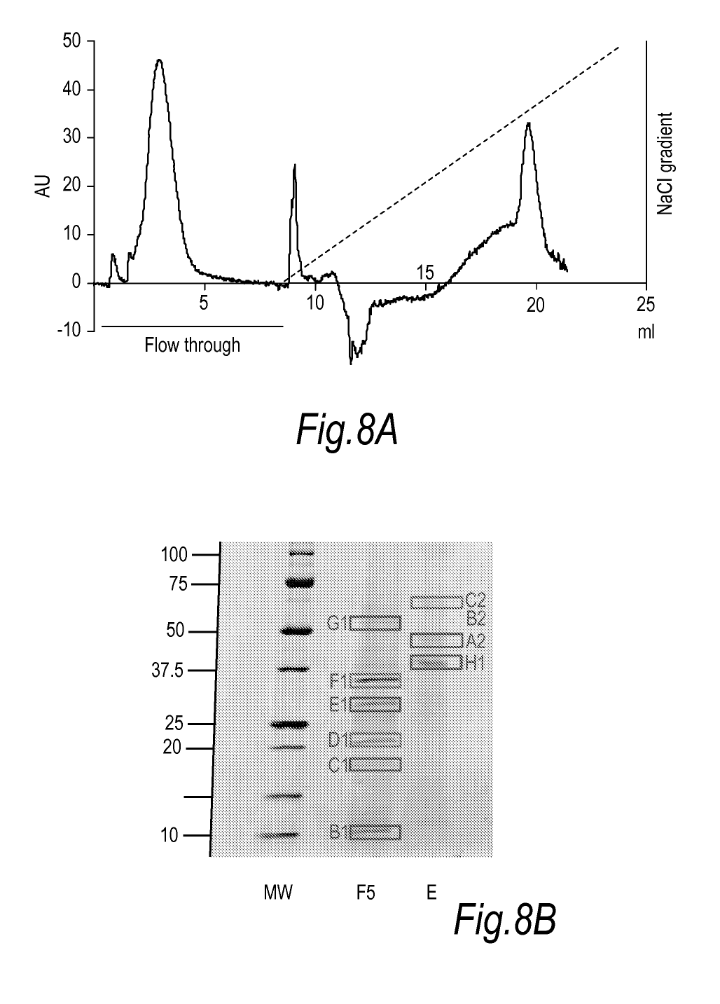

The only putative ligand suggested for Nod2 is the MDP motif found in the proteoglycan coat of gram-positive and gram-negative bacteria. If this interaction was the initiating factor for the Nod2 LRR antimicrobial effects, preincubating the domains with bacterial components containing this motif would be expected to inhibit the antibacterial activity. A competition assay was set up using Nod2 LRR activity against S. aureus. The recombinant LRR domains or BSA (control) were preincubated with various components of the S. aureus membrane prior to addition to live S. aureus and the bacterial viability assessed as in Table 1 above (FIG. 6). Neither S. aureus proteoglycan containing the MDP motif, nor lipoteichoic acid inhibited the antibacterial effect of the Nod2 LRR domains. Only heat-killed S. aureus was capable of inhibiting the Nod2 LRR activity. This indicates that the target for the antibacterial effects of Nod2 are independent of their interaction with bacterial proteoglycan and suggest that the signalling function and antibacterial activity of Nod2 have distinct bacterial targets.

### 6.5.8 Nod2 Activity Against Gram-Negative Bacterial Efflux Pump Mutants

The target for the antimicrobial activity of Nod2 LRR domains was investigated. The activity did not appear to depend on binding to the outer membrane of bacteria (FIG. 6). Therefore, the mechanism of action for Nod2, Nod 1 and Nod2 3020insC LRR domains were investigated using efflux pump mutants of Escherichia coli, Pseudomonas aeruginosa or Haemophilus influenzae (Table 3). Efflux pump mutants reduce the concentration of intracellular molecules by pumping them from the periplasmic space across the outer membrane. Two of the efflux mutant bacteria tested (E. coli and H. influenzae) demonstrated a significant increase in sensitivity to the LRR domains of Nod2 and Nod1. These two bacteria were also more resistant to the Crohns'-associated LRR mutant of Nod2 than the wild type. This suggests that the target for the LRR domains is intracellular and more sensitive to wild type than Crohn's-associated Nod2.

TABLE 3
Nod LRR domain minimal inhibitory concentration (MIC) against aerobic
gram negative bacteria (microgram/ml).

| | Strain # | Nod1 | Nod2 | 3020 | Tetracycline |
|---|---|---|---|---|---|
| E. coli | NB27004 | >128 | >128 | >128 | 4 |
| E. coli | NB27005* | 32 | 32 | >128 | 0.5 |
| P. aeruginosa | NB52019 | >128 | >128 | >128 | 32 |
| P. aeruginosa | NB52020* | >128 | >128 | >128 | 1 |
| H. influenzae | NB65027 | >128 | >128 | >128 | 0.5 |
| H. influenzae | NB65027- | 4 | 4 | 64 | 0.5 |
| | CDS0021* | | | | |

*indicates efflux pump (TolC: E. coli, H. influenzae; mexAB/oprM: P. aeruginosa) mutant strain

### 6.5.9 Identification, Partial Purification and Potential Identification of Nod2 Antibacterial Target by Mass Spectrometry.

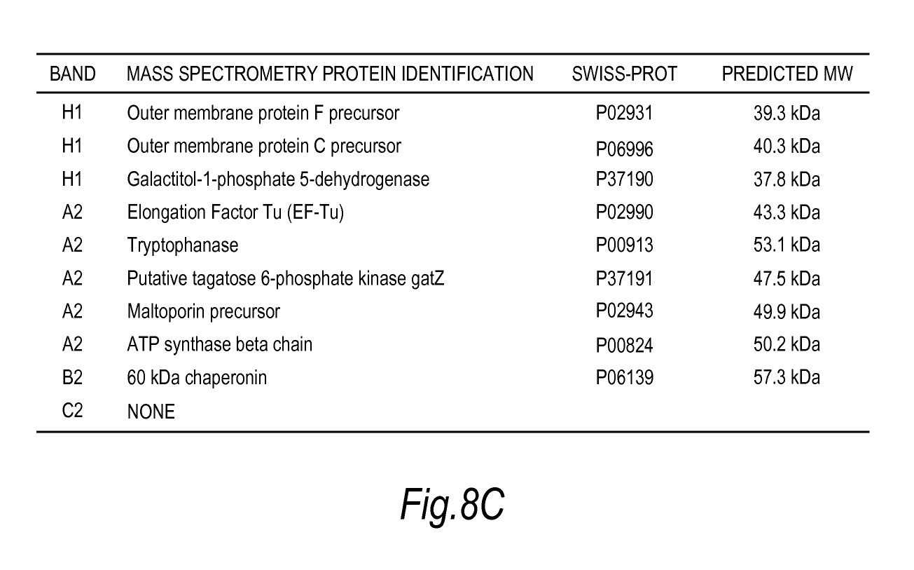

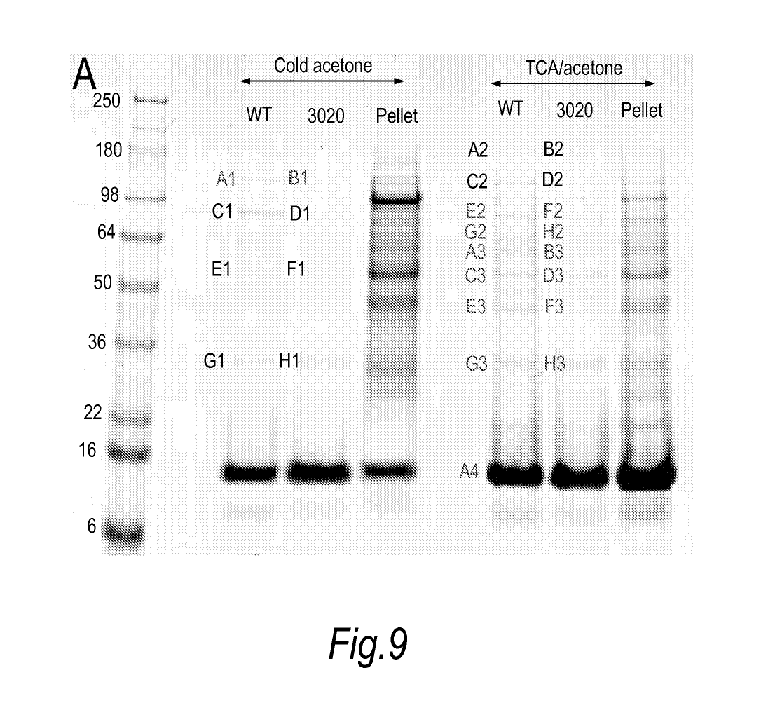

The evidence presented suggests that Nod2 has direct antibacterial activity by binding to an intracellular bacterial target via its LRR domain. In addition, the Crohn's-associated Nod2 mutation 3020insC is deficient in its antimicrobial activity. A series of experiments were conducted to identify the Nod2 bacterial target mediating the antimicrobial activity of the LRR domain. E. coli were fractionated sequentially by French press, detergent and guanidinium HCl and assessed by a competition assay to find fractions that inhibited S. aureus killing by Nod2 LRR (FIG. 7). The inhibitory fraction initially found in the detergent insoluble membrane fraction of E. coli, was solubilised in guanidinium HCl and fractionated by gel filtration. Fraction 5 from the gel filtration contained a protein that inhibited Nod2 LRR antimicrobial activity against S. aureus as demonstrated by the activity detected following treatment of the fraction with proteinase K. This fraction was loaded onto a Nod2 LRR affinity column and associated proteins eluted by a NaCl gradient (FIG. 8). Eluted proteins were separated by SDS-PAGE, bands extracted and proteins identified by mass spectrometry. The major eluted band (FIG. 8, panel B, band H1) contained two outer membrane proteins (porins) OmpF and OmpC. These proteins are found on the outer membrane of gram-negative bacteria and permit the entry of peptides into the periplasmic space of bacteria (REFERENCE). The porins are likely the first point of contact of the Nod2 LRR domains and allow their penetration into the periplasmic space of gram-negative bacteria. Taken in the context of the enhanced efficacy of LRR domains against efflux pump mutants of E. coli (Table 3) it is likely that the porins are not the target per se, but are involved in the antimicrobial mechanism by serving as a point of entry into the bacteria. In addition, gram-positive bacteria do not generally express porins, yet are sensitive to Nod LRR domains suggesting that this is not the ultimate target of Nod2 antibacterial activity.

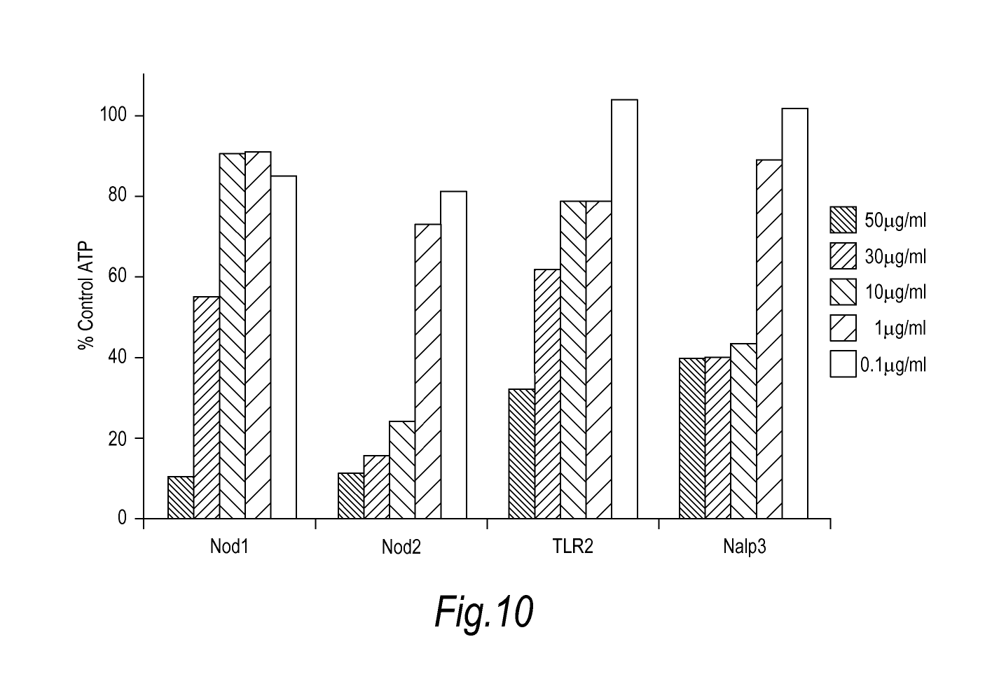

In order to identify the putative intracellular target, the entire detergent-insoluble fraction from E. coli was extracted with guanidinium HCl, split into two fractions and the fractions loaded on either 1) a Nod2 LRR domain affinity column or 2) a Nod2 LRR 3020insC domain affinity column. The proteins that bound to either column were analysed by mass spectrometry following SDS-PAGE (FIG. 9). The porins were identified again in bands C3 and E3 (bands indicated in FIG. 9). Table 4 lists all of the proteins identified in bands E3 eluted from the Nod2 LRR affinity column and band F3 from the Nod2 LRR 3020insC affinity column. Notably, the porins (OmpC and OmpF) were specifically identified in the eluate from the WT LRR column but not the 3020insC LRR column. This indicates that the Crohn's-mutant may not gain access to the intracellular bacterial compartment. Table 5 lists all of the proteins specifically identified with either the WT or 3020insC affinity column. As indicated, OmpC and OmpF were specifically demonstrated to associate with the WT LRR affinity column. Other specific proteins were also identified, some of which are demonstrated to be essential for normal E. coli growth. Therefore, several putative candidates for the Nod2 LRR antimicrobial target have been identified.

TABLE 4
Mass spectrometry identification of OmpC and OmpF in WT, but not
3020insC LRR domain affinity-purified proteins from the detergent-insoluble E.coli
fraction.

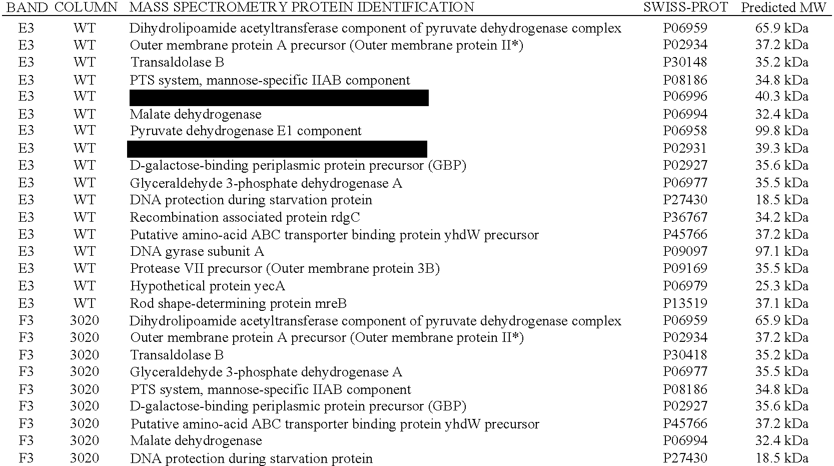

E3 and F3 indicate bands excised as indicated in FIG. 9.

TABLE 5
Mass spectrometry identification of E. coli proteins specifically
associated with the WT or 3020insC LRR-domain.

| BAND | COLUMN | MASS SPECTROMETRY PROTEIN IDENTIFICATION | SWISS-PROT | Predicted MW |
|---|---|---|---|---|
| E2 | WT | Thiamine biosynthesis protein thiC | P30136 | 71.3 kDa |
| G2 | WT | Glucose-6-phosphate isomerase | Q8FB44 | 61.6 kDa |
| A3 | WT | Aminoacyl-histidine dipeptidase | P15288 | 52.9 kDa |
| A3 | WT | Transcription termination factor rho | P03002 | 47.0 kDa |
| A3 | WT | Nitrogen regulation protein | P06713 | 52.3 kDa |
| A3 | WT | ATP synthase alpha chain | P00822 | 55.4 kDa |
| C3 | WT | Hypothetical protein ycfD | P27431 | 42.6 kDa |
| C3 | WT | Peptidase T | P29745 | 45.1 kDa |
| C3 | WT | Outer membrane protein C precursor (Porin ompC) | P06996 | 40.3 kDa |
| E3 | WT | Outer membrane protein C precursor (Porin ompC) | P06996 | 40.3 kDa |
| E3 | WT | Outer membrane protein F precursor (Porin ompF) | P02931 | 39.3 kDa |
| E3 | WT | Recombination associated protein rdgC | P36767 | 34.2 kDa |
| E3 | WT | Protease VII precursor (Outer membrane protein 3B) | P09169 | 35.5 kDa |
| E3 | WT | Rod shape-determining protein mreB | P13519 | 37.1 kDa |
| G3 | WT | Ribonuclease I precursor | P21338 | 30.0 kDa |
| G3 | WT | GrpE protein (HSP-70 cofactor) | P09372 | 21.7 kDa |
| G3 | WT | Hypothetical amino-acid ABC transporter ATP-binding protein yhdZ | P45769 | 28.8 kDa |
| G3 | WT | 3-methyl-2-oxobutanoate hydroxymethyltransferase | P31057 | 28.3 kDa |
| B1 | 3020 | ClpB protein (Heat shock protein F84.1) | P03815 | 95.7 kDa |
| F2 | 3020 | Formate acetyltransferase 1 | P09373 | 85.4 kDa |
| H2 | 3020 | Glucans biosynthesis protein G precursor | P33136 | 57.7 kDa |
| H2 | 3020 | Glutamine synthetase | P06711 | 51.9 kDa |
| H2 | 3020 | Pyruvate kinase I | P14178 | 51.4 kDa |
| B3 | 3020 | Glycerol kinase | P08859 | 56.3 kDa |
| H3 | 3020 | Single-strand binding protein (SSB) | P02339 | 18.8 kDa |

Proteins were identified from the 1% triton-insoluble fraction of E. coli extracted with GuHCl. Bands indicated correlate with those identified in FIG. 3-9.

### 7. BIBLIOGRAPHICAL INFORMATION

Alder M N, Rogozin I B, Iyer L M, Glazko G V, Cooper M D, Pancer Z (2005) Diversity and function of adaptive immune receptors in a jawless vertebrate. Science; 310: 1970-1973.

Barnich N, Aguirre J E, Reinecker H C, Xavier R and Podolsky D K (2005) Membrane recruitment of Nod2 in intestinal epithelial cells is essential for nuclear factor-kB activation in muramyl dipeptide recognition. J. Cell Biol.; 170: 21-26.

Barnich N, Hisamatsu T, Aguirre J E, Xavier R, Reinecker H C, Podolsky D K (2005) GRIM-19 interacts with nucleotide oligomerization domain 2 and serves as downstream effector of anti-bacterial function in intestinal epithelial cells. J. Biol. Chem.; 280: 19021-19026.

Dangl J L, Jones J D G (2001) Plant pathogens and integrated defence responses to infection. Nature; 411:826-833.

Darfeuille-Michaud A (2002) Adherent-invasive Escherichia coli: a putative new E. coli pathotype associated with Crohn's disease. Int. J. Med. Microbiol.; 292: 185-193.

Hampe J, Franke A, Rosenstiel P, Till A, Teuber M, Huse K, Albrecht M, Mayr G, De la Vega F M, Briggs J, Gunther S, Prescott N J, Onni C M, Hasler R, Sipos B, Folsch U R, Lengauer T, Platzer M, Mathew C G, Krawczak M, Schreiber S (2007) A genome-wide association scan of nonsynonymous SNPs identifies a susceptibility variant for Crohn disease in ATG16L1. Nature Genetics; 39: 207-211.

Hisamatsu T, Suzuki M, Reinecker H C, Nadeau, McCormick B A, Podolsky D K (2003) Card15/Nod2 functions as an antivacterial factor in human intestinal epithelial cells. Gastroenterology; 124: 993-1000.

Hugot J P, Chamaillard M, Zouali H, Lesage S, Cezard J P, Belaiche J, Almer S, Tysk C, O'Morain C A, Gassull M, Binder V, Finkel Y, Cortot A, Modigliani R, Laurent-Puig P, Gower-Rousseau C, Macry J, Colombel J F, Sahbatou M, Thomas G (2001) Association of NOD2 leucine-rich repeat variants with susceptibility to Crohn's disease. Nature; 411: 599-603.

Kobayashi K S, Chamaillard M, Ogura Y, Henegariu 0, Inohara N, Nunex G, Flavell R A (2005) Nod2-dependent reguolation of innate and adaptive immunity in the intestinal tract. Science; 307: 731-734.

Libioulle C, Louis E, Hansoul S, Sandor C, Farnir F, Franchimont D, Vermeire S, Dewit O, de Vos M, Dixon A, Demarche B, Gut I, Heath S, Foglio M, Liang L, Laukens D, Mni M, Zelenika D, Van Gossum A, Rutgeerts P, Belaiche J, Lathrop M, Georges M (2007) Novel Crohn disease locus identified by genome-wide association maps to a gene desert on 5p13.1 and modulates expression of PTGER4. PLOS Genetics; 3: 0538-0543.

Liu Y, van Kruiningen H J, West A B, Cartun R W, Cortot A, Colombel J F (1995) Immunocytochemical evidence of Listeria, Escherichia coli, and Streptococcus antigens in Crohn's disease. Gastroenterology; 108: 1396-1404.

Maeda S, Hsu L C, Liu H, Bankston L A, Iimura M, Kagnoff M F, Eckmann L, Karin M (2005) Nod2 mutation in Crohn's disease potentiates NF-kB activity an IL-1B processing. Science; 307: 734-738.

Matsushima N, Tachi N, Kuroki Y, Enkhbayar P, Osaki M, Kamiya M, Kretsinger R H (2005) Structural analysis of leucine-rich-repeat variants in proteins associated with human diseases. Cell. Mol. Life. Sci.; 62: 2771-2791.

Miceli-Richard C, Lesage S, Rybojad M, Prieur A M, Manouvrier-Hanu S, Hafner R, Chamaillard M, Zouali H, Thomas G and Hugot J P (2001) Card15 mutations in Blau syndrome. Nature Genetics; 29: 19-20.

Nagawa F, Kishishita, N, Shimizu K, Hirose S, Miyoshi M, Nezu J, Nishimura T, Nishizumi H, Takahashi Y, Hashimoto S I, Takeuchi M, Miyajima A, takemori T, Otsuka A J, Sakano H (2007) Antigen-receptor genes of the agnathan lamprey are assembled by a process involving copy choice. Nature Immunology; 8: 206-213.

Ogura Y, Bonen D K, Inohara N, Nicolae D L, Chen F F, Ramos R, Britton H, Moran T, Karaliuskas R, Duerr R H, Achkar J P, Brnat S R, Bayless T M, Kirschner B S, Hanauer S B, Nunez G, Cho J H (2001) A frameshift mutation in NOD2 associated with susceptibility to Crohn's disease. Nature; 411:603-606.

Pancer Z, Amemiya C T, Ehrhardt G R A, Ceitlin J, Gartland G L, Cooper M D (2004) Somatic diversification of variable lymphocyte receptors in the agnathan sea lamprey. Nature; 430: 174-180.

Raelson J V, Little R D, Ruether A, Fournier H, Paquin B, Van Eirdewegh P, Bradley W E C, Croteau P, Nguyen-Huu Q, Segal J, Debrus S, Allard R, Rosenstiel P, Franke A, Jacobs G, Nikolaus S, Vidal J M, Szego P, Laplante N, Clark H F, Paulussen R J, Hooper J W, Keith T P, Belouchi A, Schreiber S (2007) Genome-wide association study for Crohn's disease in the Quebec Founder Population identifies multiple validated disease loci. Proc. Natl. Acad. Sci. USA; 104: 14747-14752.

Swidsinski A, Ladhoff A, Pernthaler A, Swidsinski S, Loening-Baucke V, Ortner M, Weber J, Hoffmann U, Schreiber S, Dietel M, Lochs H (2002) Mucosal flora in inflammatory bowel diseases. Gastroenterology; 122: 44-54.

Ting J P Y, Davis B K (2005) Caterpiller: a novel gene family important in immunity, cell death, and diseases. Annu. Rev. Immunol.; 23: 387-414.

Travassos L H, Carneiro L A M, Girardin S E, Boneca I G, Lemos R, Bozza M T, Domingues R C P, Coyle A J, Bertin J, Philpott D J, Plotkowski M C (2005) Nod1 participates in the innate immune response to Pseudomonas aeruginosa. J. Biol. Chem.; 280: 36714-36718.

1. An isolated protein comprising a plurality of LRR (leucine rich repeat) domains, for use as an antimicrobial agent.

2. The protein of claim 1, wherein the C-terminus of the protein is an LRR domain.

3. The protein of claim 1, wherein each LRR domain independently consists essentially of an amino acid sequence of formula (I):
(F1LxxLxL(xxZ)_{Y}F2) (I)
wherein:
F1 and F2 are independently, a contiguous amino acid sequence of between 1 and 30 residues;
x can be any amino acid;
L can be Leu, Ile, Val or Phe;
Z can be NxL or CxxL;
N is Asn, Thr, Ser or Cys;
C is Cys or Ser; and
Y=0 or 1.

4. The protein of claim 3, wherein at least 2 L residues in each LRR are Leu.

5. The protein of claim 4, wherein at least 3 L residues in each LRR are Leu.

6. The protein of claim 1, comprising at least 3 LRR domains.

7. The protein of claim 1, comprising at least 4 LRR domains.

8. The protein of claim 1, comprising at least 5 LRR domains.

9. The protein of claim 1, comprising at least 6 LRR domains.

10. The protein of claim 1, wherein the protein is antibacterial.

11. The protein of claim 10, for use on gram positive bacteria.

12. The protein of claim 10 for use on gram negative bacteria.

13. The protein of claim 10, for treatment of bacterial infection in a human.

14. The protein of claim 1, which is selected from the group consisting of NOD, TLR, CIITA.

15. The protein of claim 14, wherein the NOD is NOD1 or NOD2.

16. The protein of claim 14, wherein the TLR is selected from the group consisting of; TLR1, TLR2, TLR3, TLR4, TLR5TLR6, TLR7, TLR8, TLR9, TLR10, TLR11, TLR12, TLR13.

17. The protein of claim 16, wherein the TLR is TLR2, TLR4 or TLR5.

18. The protein of claim 1, wherein the protein has direct antimicrobial activity per se.

19. The protein of claim 18, wherein the direct antimicrobial activity is effective under in vitro conditions.

20. The protein of claim 1, comprising 5 or more LRR (leucine rich repeat) domains, for use as an antibacterial agent, wherein the C-terminus of the protein is an LRR domain and each LRR domain independently comprises an amino acid sequence of formula (I):
(F1LxxLxL(xxZ)_{Y}F2) (I)
wherein:
F1 and F2 are independently, a contiguous amino acid sequence of between 1 and 30 residues;
x can be any amino acid;
L can be Leu, Ile, Val or Phe;
Z can be NxL or CxxL;
N is Asn, Thr, Ser or Cys;
C is Cys or Ser; and
Y=0 or 1.

21. The protein of claim 20, wherein at least 2 L residues in each LRR are Leu.

22. A pharmaceutical composition comprising an isolated protein according to claim 1.

23. The pharmaceutical composition of claim 20, for use as an antimicrobial.

24. The pharmaceutical composition of claim 22, for use in treating and/or preventing bacterial infection in a host susceptible to infection.

25. The composition of claim 24 wherein the host is a mammal such as human.

26. The composition of claim 24, wherein the human is afflicted with a gastrointestinal disease such as Crohns disease, IBD or IBS.

27. A method of treating a microbial infection in a human comprising administering to that human an effective amount of an isolated protein comprising a plurality of LRR (leucine rich repeat) domains.

28. The method of claim 27, wherein the C-terminus of the protein is an LRR domain.

29. The method of claim 27, wherein each LRR domain independently consists essentially of an amino acid sequence of formula (I):
(F1LxxLxL(xxZ)_{Y}F2) (I)
wherein:
F1 and F2 are independently, a contiguous amino acid sequence of between 1 and 30 residues;
x can be any amino acid;
L can be Leu, Ile, Val or Phe;
Z can be NxL or CxxL;
N is Asn, Thr, Ser or Cys;
C is Cys or Ser; and
Y=0 or 1.

30. The method of claim 29, wherein at least 2 L residues in each LRR are Leu.

31. The method of claim 29, wherein at least 3 L residues in each LRR are Leu.

32. The method of claim 27, wherein the protein comprises at least 3 LRR domains.

33. The method of claim 27, wherein the protein comprises at least 4 LRR domains.

34. The method of claim 27, wherein the protein comprises at least 5 LRR domains.

35. The method of claim 27, wherein the protein comprises at least 6 LRR domains.
